(19) United States
(12) Patent Application Publication
(10) Pub. No.: US 2016/0022204 A1
(43) Pub. Date: Jan. 28, 2016

(54) AN APPARATUS FOR REMOTE CONTACTLESS MONITORING OF SLEEP APNEA

(71) Applicant: Kirill MOSTOV, Berkeley, CA (US)

(72) Inventor: Kirill Mostov, Berkeley, CA (US)

(21) Appl. No.: 14/775,639

(22) PCT Filed: Mar. 12, 2014
(86) PCT No.: PCT/US14/25082

§ 371 Date: Sep. 11, 2015

Related U.S. Application Data
(60) Provisional application No. 61/779,914, filed on Mar. 13, 2013.

(51) Int. Cl. A61B 5/00; A61B 7/00; A61B 5/1455; A61B 5/05

(52) U.S. Cl. CPC A61B 5/4818; A61B 5/05; A61B 7/003; A61B 5/7282; A61B 5/14552; A61B 5/0013; A61B 5/6826; A61B 2562/0219

(57) ABSTRACT

An apparatus for remote contactless monitoring of sleep apnea, comprising a radar transmitter having a transmitting antenna for radiation of radio frequency signal towards a human body, and a radar receiver for receiving a signal reflected from the human body. The radar receiver comprises a receiving antenna positioned at a predefined distance from the transmitting antenna. The apparatus further comprises an accelerometer adapted to be placed on a human body, a microphone and a signal processor. Respective outputs of the radar receiver, accelerometer and microphone are connected to the input of the signal processor which is configured for extracting and processing apnea-specific physiological parameters of the at least one human body from the inputted signals of the receiver, accelerometer, and microphone.

### RELATED APPLICATIONS

This application claims priority from U.S. Provisional Patent Application No. 61/779,914 entitled “A Device for Remote Contactless Monitoring of Sleep Apnea”, filed 13 Mar. 2013, which is incorporated herein by reference.

### FIELD OF THE INVENTION

This invention relates to devices for remote contactless monitoring of sleep apnea.

### BACKGROUND OF THE INVENTION

Status monitoring of patients with respiratory disturbances during sleep is an important and complicated task. This is explained by the fact that the sleep apnea represents a high level of risk for patient's life. From the other side, the sleep apnea detection requires permanent monitoring an individual state, and thus can be effectively attained by automatic equipment. In addition, such equipment shall be arranged in a most convenient way for patients and provide reliable and accurate information.

Previous attempts to monitor sleep apnea of a patient were made by means of expensive and clumsy devices. Multiple sensors have been positioned on a patient body; often these caused wire mishmash. Due to these factors, such devices could not provide reliable information, and were inconvenient for patients and for the staff.

A prior medical device (U.S. Pat. No. 6,062,216 by Corn) uses a laser radar whose beam is directed upon a patient. A signal reflected from a patient's body is captured by a receiver. A signal processor calculates the range to a patient and the range rate of the patient, using a reference signal from the transmitter. A Doppler analysis of the reflected signal is implemented by applying the fast Fourier transform to measurement signals which are obtained by mixing light signals, such as the illumination source and the collected return image light, on a photo-detector.

The analysis provides information about the inhalation and exhalation phases of the patient's breathing. Upon the detection of the cessation of breathing, depending on the embodiment of this device, either an acoustic stimulus signal is generated through an auditory prompter inserted into a patient's ear, or an alarm signal is transferred to the medical personnel.

The medical device discussed above, although having the ability to detect the sleep apnea, has serious drawbacks. These include:

inability to monitor several subjects,

a special requirement to provide reflecting elements on a subject clothes, otherwise the optical reflection is inefficient,

high cost of optical elements included in the device,

susceptibility to omit apnea detection due to the lack of information and noise inside the reflected signal.

The objective of the present invention is to provide an apparatus for remote sleep apnea monitoring of one or several individuals, capable of monitoring several subjects and free from expensive components such as optics. Another objective is to provide a compact apparatus, which can be hand-held, not requiring complicated connection and installation.

### SUMMARY

The invention relates to an apparatus for remote monitoring of sleep apnea, comprising a radar transmitter having a transmitting antenna for radiation of radio frequency signal towards at least one human body, and at least one radar receiver for receiving a signal reflected from the at least one human body, the radar receiver comprising a receiving antenna positioned at a predefined distance from the transmitting antenna. The apparatus further comprises at least one accelerometer adapted to be placed on a human body, at least one microphone, and a signal processor, wherein respective outputs of the radar receiver, accelerometer and microphone are connected to the input of the signal processor which is configured for extracting and processing apnea-specific physiological parameters of the at least one human body from the inputted signals of the receiver, accelerometer, and microphone. This embodiment allows for monitoring the position and the sleep apnea of an individual who may stay motionless or move while sleeping.

Preferably, the accelerometer comprises a wireless transmitter, the signal processor comprises a wireless receiver, and the output of the accelerometer is connected to the input of the said signal processor through a wireless communication channel. Thus, the accelerometer can be connected to the signal processor through a wireless channel. This provides the convenience for the patient and excludes damaging the communication wires.

The present apparatus can comprise at least one optical video camera and at least one pulse oximeter, wherein the optical input of the video camera is directed to the at least one human body, the pulse oximeter is adapted to be positioned on the at least one human body, and the outputs of the video camera and pulse oximeter are connected to respective inputs of the signal processor. This embodiment has the highest reliability of the sleep apnea detection due to the patient's image processing and measurement of the blood oxygen level.

Preferably, the pulse oximeter is adapted to be positioned on a human finger. Further, the pulse oximeter can have a wireless transmitter, and therefore can be connected with the signal processor through a wireless channel. This embodiment provides complete remote operation.

To monitor more than one subject the apparatus can comprise at least two radar receivers, wherein their respective receiving antennae are positioned at a predetermined distance from one another and from the transmitting antenna. Each radar receiver can comprise a clocked amplifier having its input connected to the receiving antenna.

Preferably, the transmitter is adapted to produce frequency modulated signals in the form of a train of pulses with a predefined delay between the pulses.

### BRIEF DESCRIPTION OF DRAWINGS

FIG. 1 is a general block diagram of a sleep apnea monitor (SAM), according to one embodiment.

FIG. 2 is a diagram of the SAM main operation stages for sleep apnea detection, according to one embodiment.

FIG. 3 explains a signal processing procedure for determination of the range to the patient, according to one embodiment.

FIG. 4 illustrates the result of processing of a signal reflected from multiple objects, according to one embodiment.

FIG. 5 explains a signal processing procedure for determination of the patient azimuth, according to one embodiment.

FIG. 6 explains the use of triangulation method for determination of patient's azimuth, according to one embodiment.

FIG. 7 illustrates a typical signal when determining heart and respiration rates, according to one embodiment.

FIG. 8 explains a sleep apnea symptom detection algorithm based on the current respiration rate, according to one embodiment.

FIG. 9 explains a sleep apnea symptom detection algorithm based on the current amplitude of breathing, according to one embodiment.

FIG. 10 explains a sleep apnea symptom detection algorithm by analyzing changes in the heart rate, according to one embodiment.

FIG. 11 illustrates typical signals, which provide identification of various apnea types.

FIG. 12 illustrates a typical signal that makes it possible to identify limb movements.

FIG. 13 explains a method of sleep apnea monitor application in a therapy system.

FIG. 14 illustrates a general block diagram of a sleep apnea monitor with a controlled key and explains how gating is used to decrease the device's radiated power.

FIG. 15 illustrates a general block diagram of a sleep apnea monitor with a digital to analog converter installed between a computing module and a transmitter and explains how converter is used to decrease the device's radiated power.

FIG. 16 illustrates a general block diagram of a sleep apnea monitor with a digital to analog converter installed between a computing module and a control circuit of frequency deviations and explains the method how controlling the radiated signal modulation frequency is used to decrease the radiated signal power.

FIG. 17 illustrates a general block diagram of a sleep apnea monitor with an aggregate of components for decreasing the radiated signal power.

FIG. 18 illustrates a general block diagram of a SleepID device.

FIG. 19 illustrates the structure of a Data Processing Unit.

FIG. 20 illustrates the structure of a Radar Data Processing Unit.

FIG. 21 illustrates the structure of a Motion and Breathing Signal Formation Unit.

FIG. 22 illustrates the structure of an Apnea Pattern Search Unit.

FIG. 23 illustrates the structure of a Sleep/Wake Determination Unit.

FIG. 24 illustrates determination of Sleep and Wake Modes.

FIG. 25 illustrates the structure of a Microphone Data Processing Unit.

FIG. 26 shows a diagram presenting an Example of an input signal fragment.

FIG. 27 a to f shows diagrams illustrating Snoring Occurrence Detection:

a) signal energy;
b) zero crossing rate;
c) detected occurrences;
d) detected snoring occurrences;
e) snoring occurrences detected by a reference device;
f) projection of the vector of characteristic values onto a determined subset.

FIG. 28 illustrates the structure of an Optics Data Processing Unit.

FIG. 29 illustrates the structure of a Pulse Oximeter Data Processing Unit.

FIG. 30 illustrates the structure of a Joint Decision-Making Unit.

FIG. 31 shows the Doppler Module Block Diagram.

FIG. 32 is the diagram of the modified SAM main operation stages for sleep apnea detection, wherein the radar operates in a self-adaptable mode.

FIG. 33 is the general block diagram of the sleep apnea monitor (SAM) based on the ultra wide bandwidth (UWB) radar according to a preferable embodiment.

FIG. 34 is an operational flow diagram of a remote detection and monitoring apparatus based on the ultra wide bandwidth radar technology.

### DETAILED DESCRIPTION

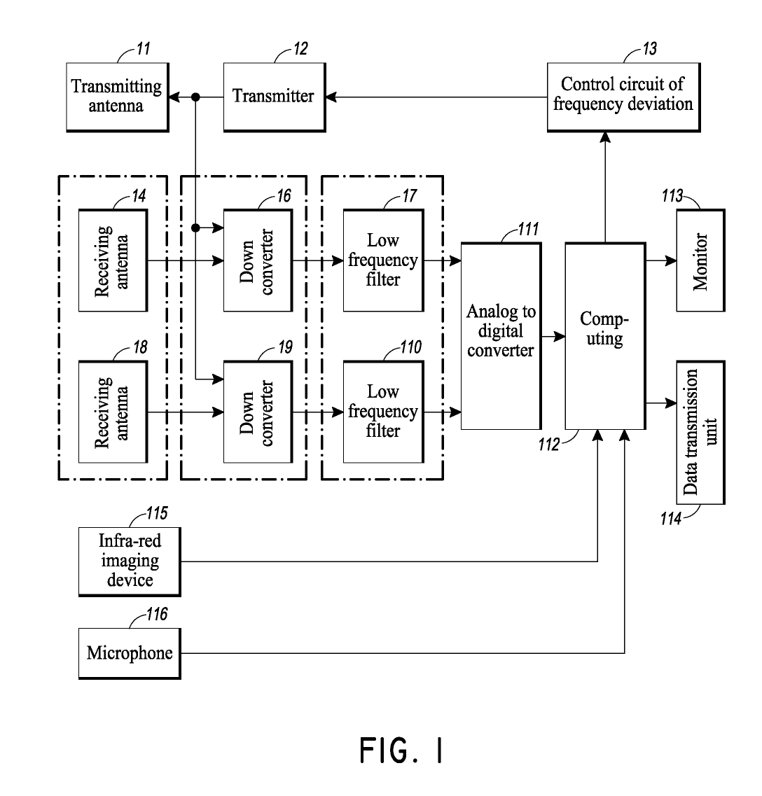

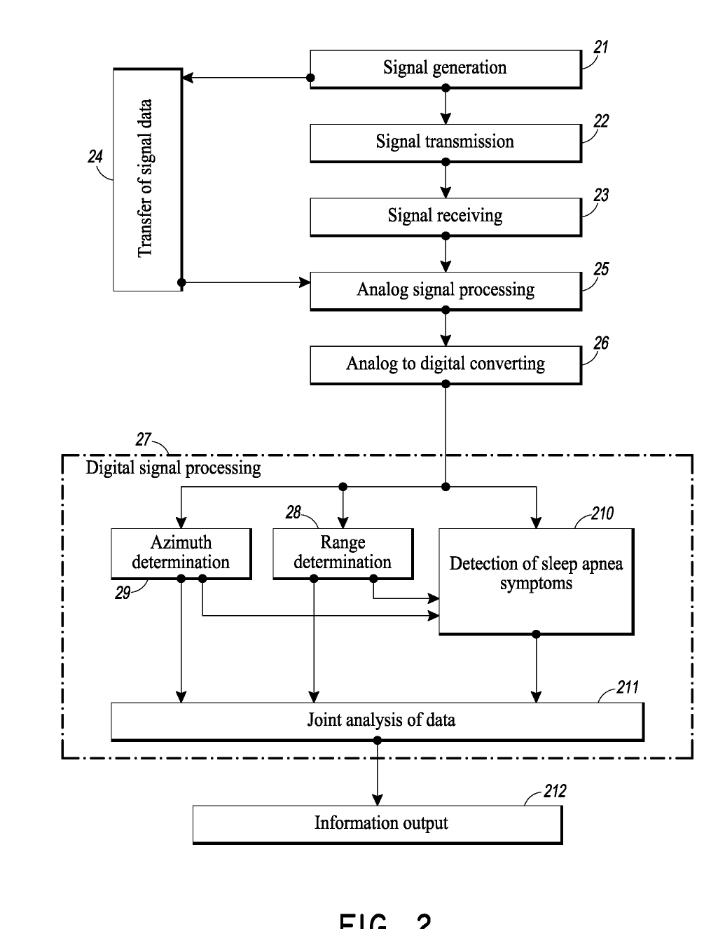

According to one embodiment, in FIG. 1 a block diagram of an exemplary sleep apnea measurement device is illustrated. The device generally includes a transmitter (12), a transmitting antenna (11), a receiver comprising two receiving antennae (14, 18), two down converters (16, 19), two low frequency filters (17, 110), analog to digital converter (111), a processor or computing module (112), control circuit of frequency deviation (13) and monitor (113).

Further, the device is equipped with an infrared imaging device 115 and a microphone 116 to collect additional information about the subject.

The device can make use of wireless communication technologies for transmission monitoring results to Sleep Apnea network operations center. To this end, the device is equipped with a data transmission unit 114. Processing monitoring results to a network operations center makes it possible to control a group of multiple devices simultaneously or transfer monitoring data to a remote monitoring station.

The transmitter (12) generates a signal that is radiated by the transmitting antenna (11), and sends the signal data to the down converters (16, 19). The signal reflected from an exposed object or human body is received by the receiving antenna_1 (14) and receiving antenna_2 (18) and is brought to the down converters (16, 19) via individual channels. Each down converter multiplies the signals that are transmitted and received in that moment. After conversion, the signals are processed by the low frequency filters (17, 110), whereby the low frequency component is isolated in each channel and then taken to the analog-to-digital converter (111). The digitized signal data is delivered to the computing module (112), which determines sleep apnea symptoms in the exposed subject and displays the information about the subject's state on the monitor screen (113).

According to one embodiment, the device detects sleep apnea symptoms in a single individual with just one receiving antenna.

When there is more than one individual within a scanned area, the device identifies which signal belongs to which target. When just one receiving antenna is used, the device is capable of determining the range to target. When there are two receiving antennae, the device is capable of determining the azimuth of a target.

Range and azimuth data make it possible to determine which signal corresponds to which subject, and separate the signals to determine the state of multiple persons at once.

A diagram of the device's main operation stages for determination a personal state is given in FIG. 2. When the device is on, the signal is generated 21, transmitted 22 and received 23. Analog processing 25 signals received via individual channels from each of the receiving antennae uses the transmitted signal data 24 obtained from the transmitter 12 FIG. 1 to the down converters 16 and 19.

When the analog signal processing in the low frequency filters is completed and the signals are digitized 26, the digital signal processing 27 takes place. The process of signal processing includes calculations that make it possible to: determine the range to the target 28, target position azimuth 29, and detect sleep apnea symptoms in the subjects 210. When state monitoring is carried out for multiple subjects, the data on the range 28 and azimuth 29 is used for signal segregation in order to detect sleep apnea symptoms 210 in individual subjects. Calculation results are jointly analyzed 211 and the information is displayed 212 on the screen.

For an object's position measurement in 2D coordinates, the range to the object's and object’ azimuth are calculated.

For range calculations, the high-frequency return signal that is received by each receiving antenna is compared against the transmitted signal with continuous frequency change modulation.

FIG. 3 explains the range finding algorithm that uses the frequency of the received signal. The transmitting antenna transmits the signal F′. The received analog signal is converted by the down converter. The low-frequency filter makes it possible to isolate the useful low-frequency component of the analog signal carrying the target range data. The analog-to-digital conversion allows the input signal to be sampled to produce a set of N points for the signal received by each antenna. At that:

$$N = ( \frac{{T}_{M}}{2} - τ ) {F}_{d}$$

wherein:

Tₘ/2=frequency sweep period: frequency change from to F1 to F2;

F_{d}=analog input sampling rate;

τ=time interval between signal transmission and signal reception.

The emitted signal F′ upon reaching the target at R distance from the device, is reflected back and subsequently received with all receiving antennas. Thus, the emitted signal will acquire a time delay: T=2*R/c, where c—the speed of light in the air. The receiver determines frequency FR, which is equal to the difference of frequencies (F′) (emitted) and (F*) (received) during the moment of time t1. Thus:

$${F}^{′} = {F}_{1} + \frac{2 ( {F}_{2} - {F}_{1} )}{{T}_{M}} {τ}_{1} ; {F}^{*} = {F}_{1} + \frac{2 ( {F}_{2} - {F}_{1} )}{{T}_{M}} ( {τ}_{1} - τ )$$

$${F}_{R} = {F}^{′} - {F}^{*} = \frac{4 ( {F}_{2} - {F}_{1} ) R}{{cT}_{M}}$$

$$R = \frac{{cT}_{M} ( {F}^{′} - {F}^{*} )}{4 ( {F}_{2} - {F}_{1} )}$$

Where R is the desired distance to the target.

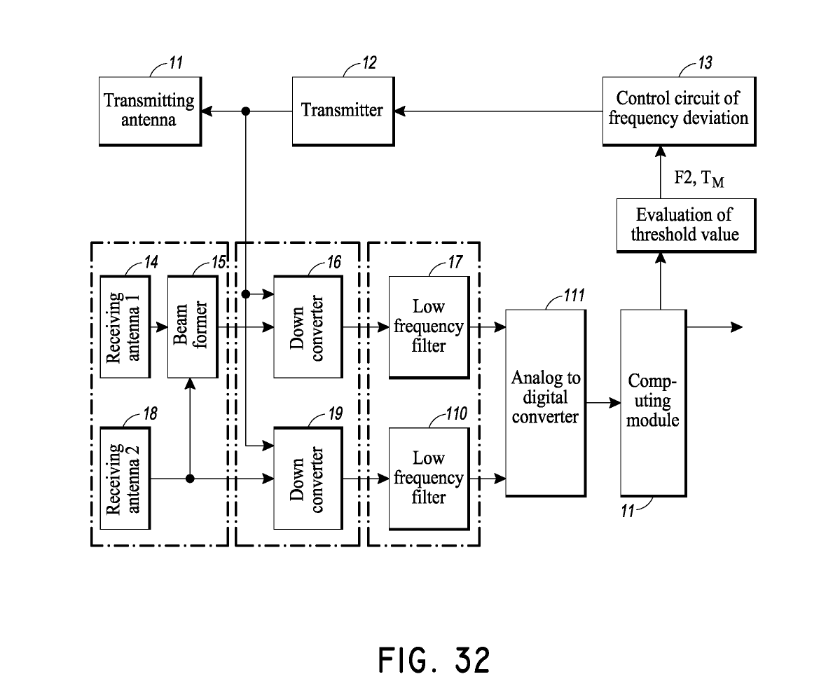

A modified SAM main operation stages for sleep apnea detection, wherein the radar operates in a self-adaptable mode are presented in FIG. 32. Here a module for evaluation of threshold value is introduced between the control circuit 13 of frequency deviation and computing module 11. This modification allows optimizing the frequency range of the radar in relation to the distance to object, and thus reduce the noise level, i.e. improve the signal-to-noise ratio.

In operation, the radar is initially in its regular mode. Computing module 13 evaluates amplitudes for each range sample. The evaluations thus obtained are delivered to the module to evaluate the threshold value. The module for evaluation threshold value, by comparing the amplitudes for each range sample, determines the sample having the biggest number (Rmax), wherein the amplitude for this sample exceeds a predetermined threshold. It is clear, however, that the maximum range to the object currently does not exceed the Rmax value. Thus, it is expedient to adjust the radar parameters F2 and TM so as to the maximum range of the radar does not exceed the Rmax value, and the frequency band is reduced proportionally. The module calculates the values F2 and TM, which are optimal in view of the Rmax value, and transmits these values to the control circuit 13 of frequency deviation. In the control circuit 13 of frequency deviation a frequency sweep for the signal emitted by the radar is provided, using the values F2 and TM determined as described above.

FIG. 4 illustrates the processing result for a signal reflected from multiple objects. When there are multiple targets within the coverage area of the device, the low-frequency signal comprises the appropriate number of harmonic components, each with a frequency corresponding to the distance to a particular target.

Azimuth determination is based on the differential diagram. For each frequency shown in FIG. 4 and corresponding to the distance to a particular target, the signal amplitude Aj acquired by the receiving antenna_1 is compared with the signal amplitude Ak acquired by the receiving antenna_2.

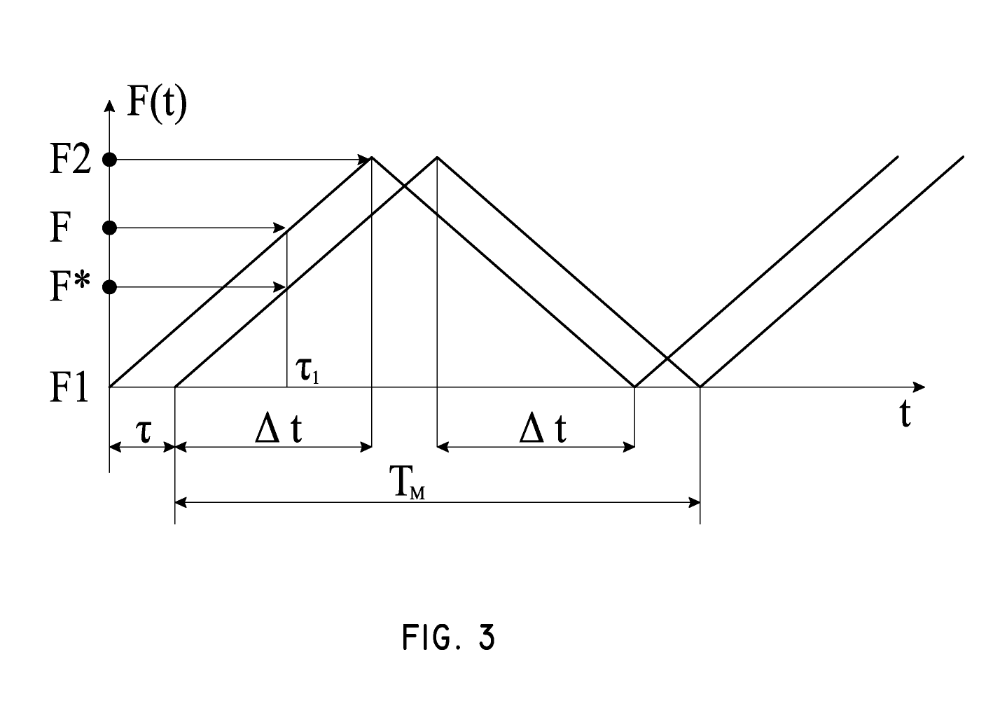

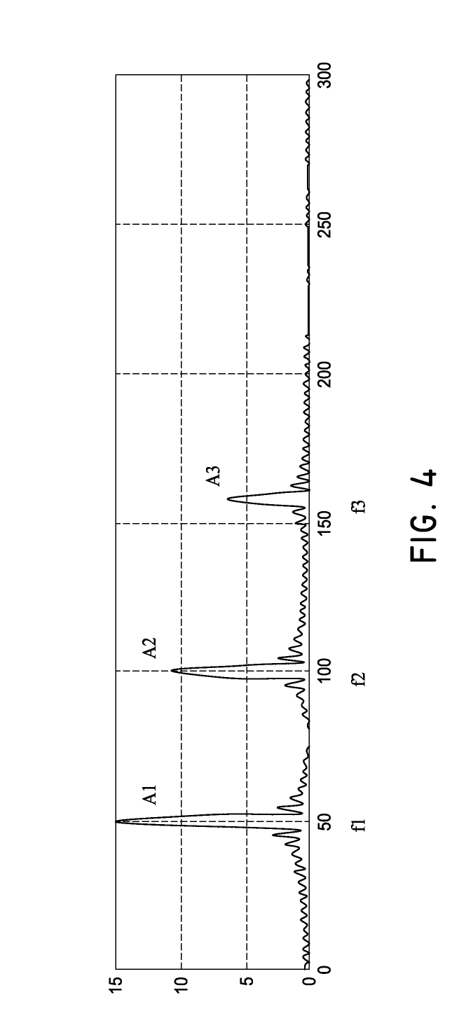

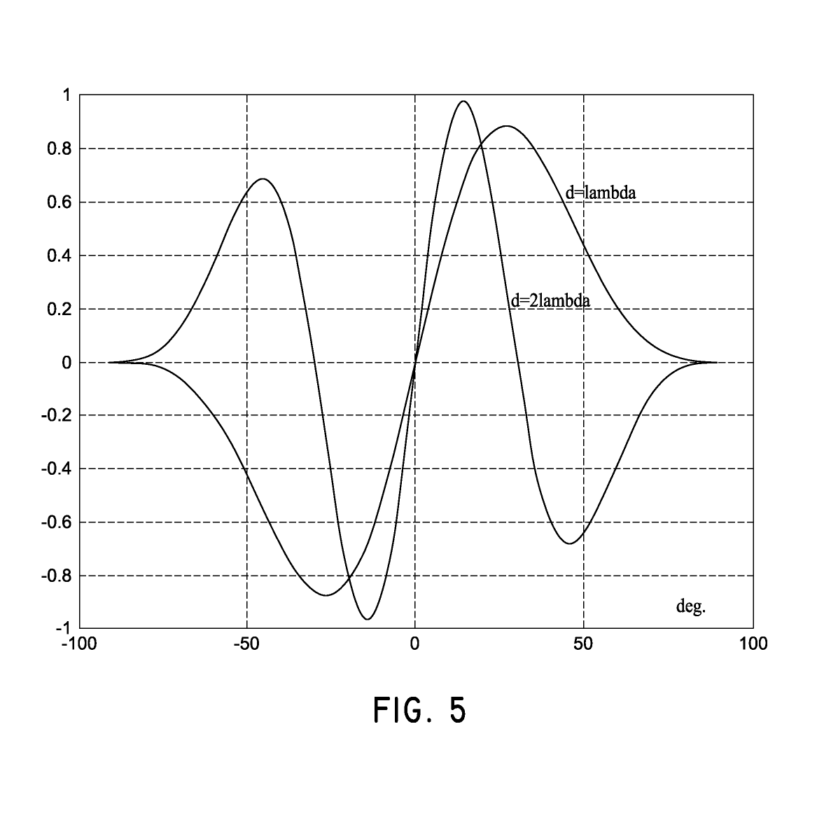

FIG. 5 demonstrates an orientation diagram of the azimuth determination. In the diagram, d is the distance between the receiving antenna_1 and the receiving antenna_2, and lambda is the wavelength. Angles between the normal and the receiving antenna_2 in degrees are plotted on the X-axis.

Within ±30° sector the value of the signal is proportional to the value of the azimuth (Plot 51). When the distance between the antennae becomes smaller, the spectrum width increases, but the slope of the curve decreases (Plot 52).

The azimuth determination is based on a turnstile characteristic P=Aⱼ/Aₖ. P defines the azimuth sign. P does not depend on the distance to the target or effective radar cross section.

Within a linear part of the orientation diagram, azimuth θ is determined as θ=S·P, where S is the slope of the curve of the turnstile characteristic.

High accuracy of the distance measurement (R.M.S. error 1 cm) allows for determining the azimuth with a desirable accuracy when the distance between antennae is about 20 cm. The distance between antennae is limited by the device dimensions.

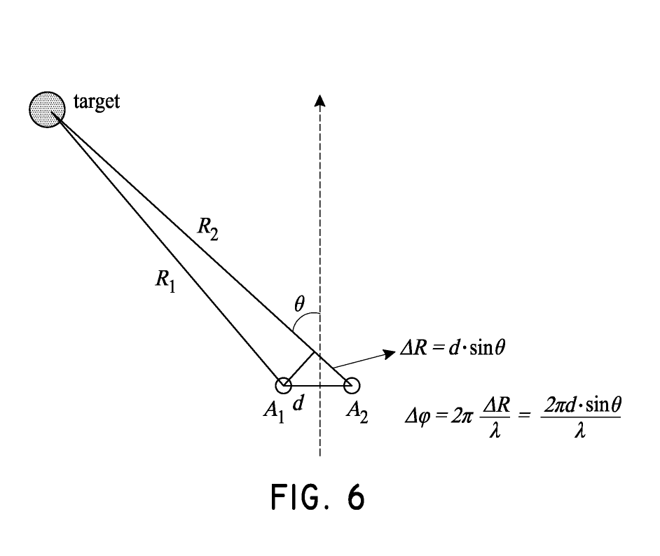

FIG. 6 explains the azimuth θ finding procedure when the triangulation method is used.

Because of the distance from antenna_1 and antenna_2 to the target (ΔR), the phase of the signal, received by receiving antenna_1, will be delayed from the phase of the signal, received by receiving antenna_2, by a value:

$$Δ ϕ = \frac{Δ R}{λ} 2 π = \frac{L \cdot sin θ}{λ} 2 π = k \cdot L \cdot sin θ ,$$

where

$$k = \frac{2 π}{λ} ,$$

λ—is the wavelength of the radiated (received) signal.

Using values of Δφ and [symbol] it is possible to determine θ.

Slow fluctuations of a probing signal allow remote measurement of different rhythms of human body functions. It is possible to measure these parameters by illuminating the entire group of people and isolating each person by his range and azimuth. For determining rhythmic processes, probing is carried out over several seconds. A series of reflected signal magnitudes are exposed to an FFT, which provides a signal pulsations spectrum for each target.

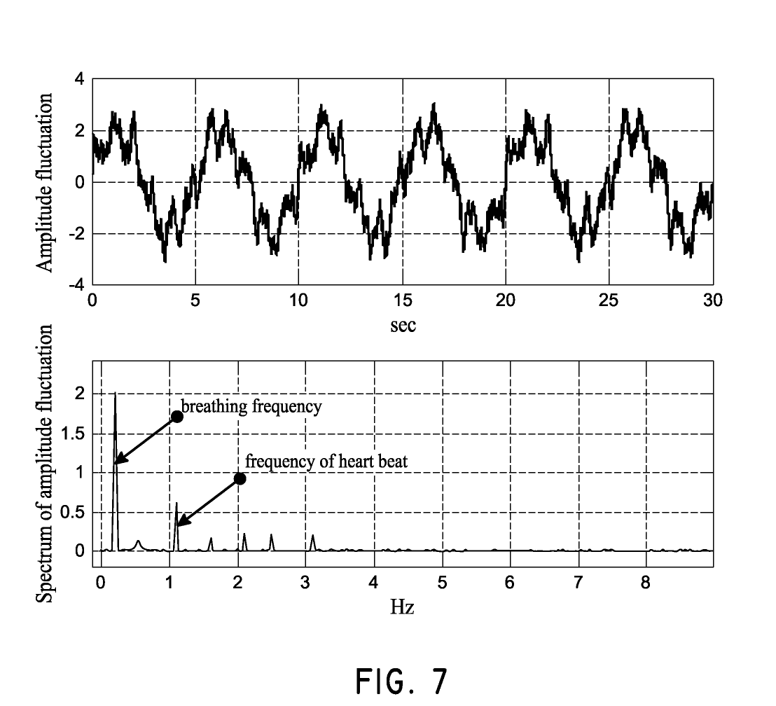

FIG. 7 demonstrates a typical signal of amplitude fluctuations caused by a person and its spectrum through analyzing the spectrum definition of physiological parameters (breathing frequency, frequency of heart beat).

The methods that the device uses to measure the rates of respiration and heartbeat are based on analyzing the changes in the distance to a subject and a subject's RCS.

Amplitude of the radar output is proportional to subject's radar cross section. On the other hand, subject's RCS is proportional to the filling of blood vessels at a rate corresponding to the heart rate, and to the saturation of blood with the oxygen at a rate corresponding to the respiration rate.

The phase variation in the signal received by the device is proportional to the variation of the distance to subject. Since the variation of the distance to subject result from body pulsation at both heartbeat and breathing rates, then it is possible to measure the heart rate and respiration rate by analyzing the signal phase change.

Based on the data about the change of patient's radar cross section, the device can determine remotely the parameters of pulsation of veins when they are filled with blood, and plot a plethysmogram.

The presence of foreign objects in the device's coverage area produces signal reflections from them. When echo signals of foreign objects are received by the device, they interfere with the desired signal and, consequently, cause calculation errors. Two receiving antennae in the device make it possible to calculate the rates of respiration and heartbeat independently on two channels. By comparing the resultant characteristics of the signals received by two antennas, the error of calculation of the monitored parameters is minimized.

Current values of patient's respiration rate and heart rate acquired during sleep monitoring are used for sleep apnea detection. Before the monitoring, the minimum allowed heart and respiration rates for normal sleep are set. These thresholds can be obtained through measurements conducted at the initial phase of the monitoring, under direct supervision of attendant staff, or taken from reference documents.

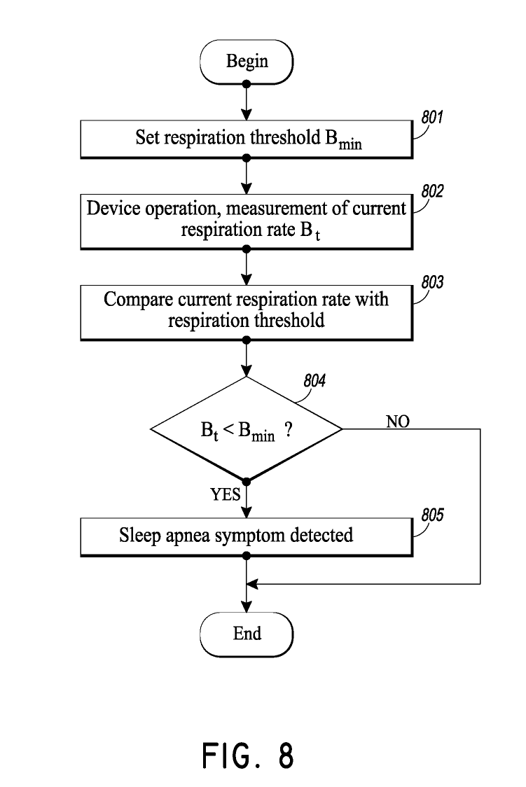

FIG. 8 explains the sleep apnea symptom detection algorithm using the current respiration rate. In the process of the device's operation, patient's current respiration rate Bt is measured 802 and compared 803 with the respiration threshold Bmin, which is set 801 in the initial step of the sleep monitoring procedure. In the result of comparison 804, if Bt<Bmin, then sleep apnea symptom is detected 805.

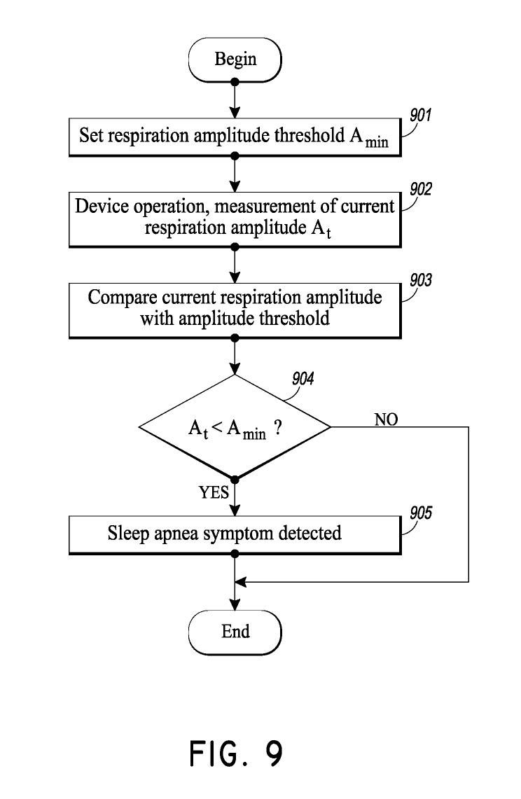

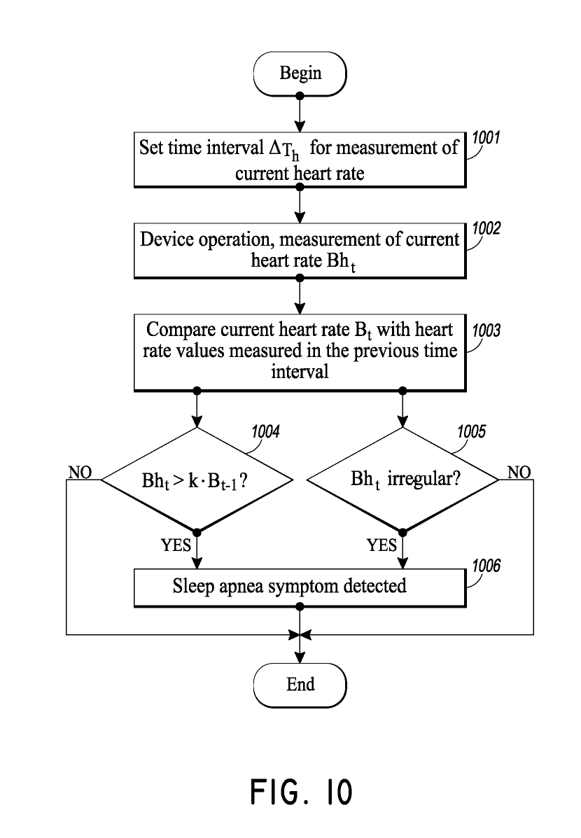

FIG. 9 explains the sleep apnea symptom detection algorithm using the current respiration amplitude. In the process of device's operation, patient's current respiration amplitude At is measured 902 and compared 903 with the amplitude threshold Amin, which is net 901 at the initial step of the sleep monitoring procedure. In the result of comparison 904, if At<Amin, then sleep apnea symptom is detected 905.

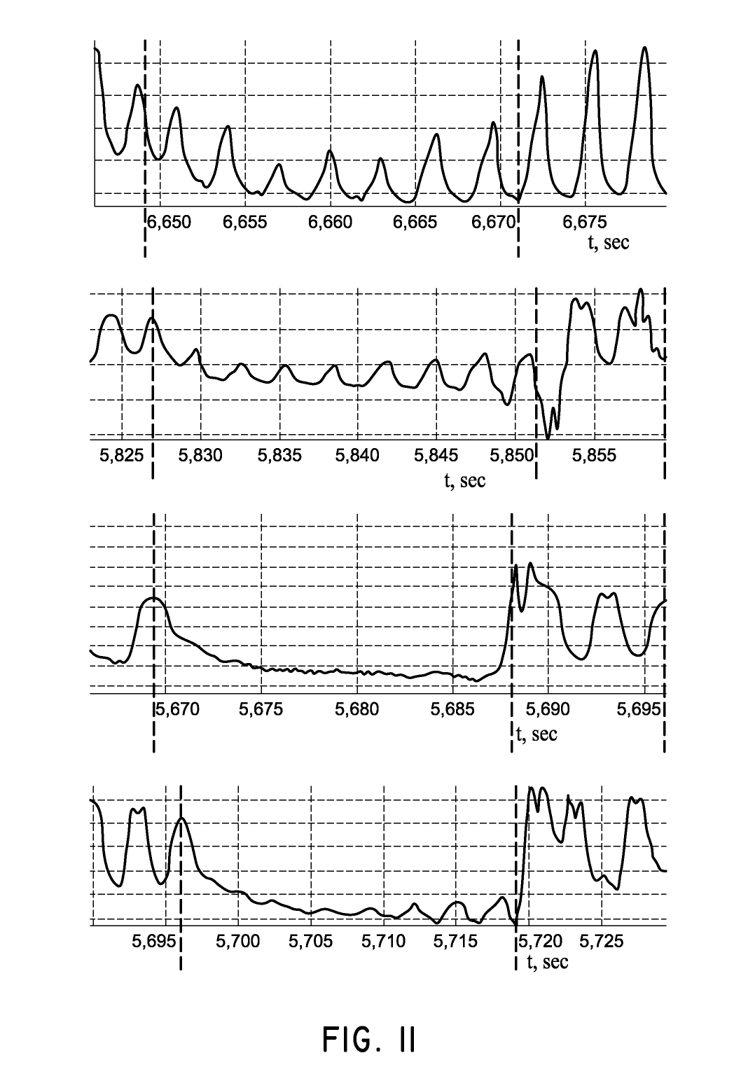

Temporary cessation of breathing in a patient may cause a sharp increase in the heart rate or irregular heartbeat. FIG. 10 explains the sleep apnea symptom detection algorithm by analyzing the heart rate change. Before starting the monitoring procedure, a time interval ΔTh is set 1001, which is used for calculation of the current heart rate. In the process of the device's operation, patient's current heart rate Bht, is measured 1002 and compared 1003 with the values obtained in the previous measurement intervals.

In the result of comparison 1004, if Bht>K·Bht−1, then the sleep apnea symptom is detected 1006. The factor K1 is set before starting the measurements; this factor is used to specify how much of a heart rate variation should be considered a considerable variation.

The heartbeat irregularity can be assessed by introducing K2 factor. In each time interval ΔTh the difference between the current heart rate and the previous heart rate is calculated, and three latest differences are saved:

ΔBhₜ=Bhₜ−Bhₜ₋₁,ΔBhₜ₋₁=Bhₜ₋₁−Bhₜ₋₂, ΔBhₜ₋₂=Bhₜ₋₂−Bhₜ₋₃

The current difference is compared against the allowed heart rate change [ΔBhₜ], which is calculated using the formula: [ΔBhₜ]=K₂Bhₜ. If in three measurement intervals in a row, and ΔBhₜ changes its sign in each measurement interval (i.e. the heart rate changes up and down), then irregular heartbeat 1005 is detected and the sleep apnea symptom is recorded 1006.

Different processes that occur during sleep have different effect on the parameters of the signal reflected from the subject. The frequency, amplitude and value of the received signal are all changed. When analyzing the signal, distinctive shape variations make it possible to identify symptoms of various apnea types.

FIG. 10 illustrates typical signal shapes during hypopnea, obstructive apnea, central apnea, mixed apnea and gasping. Hypopnea is defined as an abnormal respiratory event associated with at least a 30% reduction in thoracoabdominal movement or airflow as compared to baseline. The device is able to pick up such reduction in thoracoabdominal movement, as can be seen from the lower amplitude of the waveform. If the amplitude is even lower than in case of hypopnea, then the obstructive apnea phase can occur. The characteristic feature differentiating the obstructive apnea from the hypopnea is the presence of a specific phase of gasping.

If the signal shows almost zero amplitude, then the central apnea condition is detected. If a transition from complete cessation of breathing to a small amplitude breathing is detected between two phases of normal breathing, then the mixed apnea phase is detected. Mixed apnea is usually followed by gasping.

By analyzing the signal reflected from the subject, his or her heart rate can be determined. The device makes it possible to estimate the heart rhythm variability and determine the values of parameters such as:
1. Moda—the most common interval recorded during the monitoring period;
2. Mean—the average value of the intervals recorded during the monitoring period;
3. Number of respiratory rate intervals recorded during the monitoring period;
4. LF/HF—the ratio of average powers in the medium and high spectrum regions of the heart rhythm variation curve.
Based on a subject testing using electroencephalogram and the data acquired by the device based on a microwave radar, parameter values (Moda, Mean, etc.) can be measured for the sleep phase of a particular subject. This provides the opportunity further to identify the sleep phase just by using the standalone device.

In the determination of physiological parameters of a subject, it is desirable that the subject's body position is kept under control. Comparing the body position with the parameters of the signal reflected by the subject allows to make the diagnostics of Sleep Apnea symptoms more accurate. With a view to control subject's body position, special markers should be attached in different locations on the subject's clothes, or the subject should be wearing special underclothes (undershirt, pajamas) with such markers. The markers in different locations on the clothes should be selected to have different effects on the reflected signal parameters.

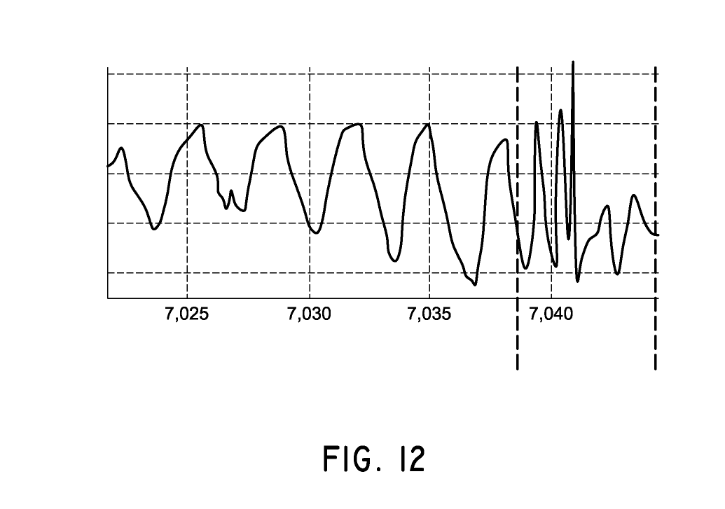

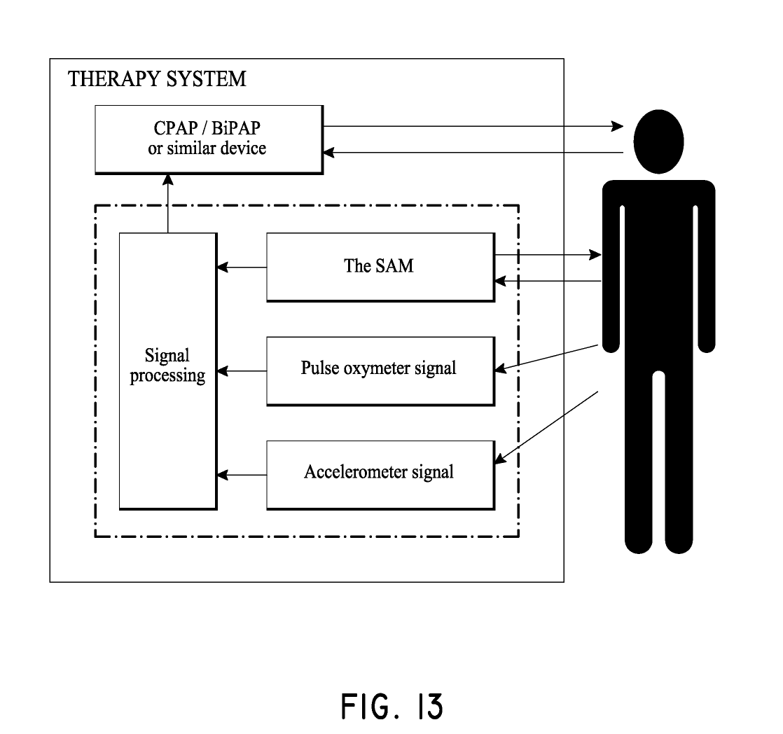

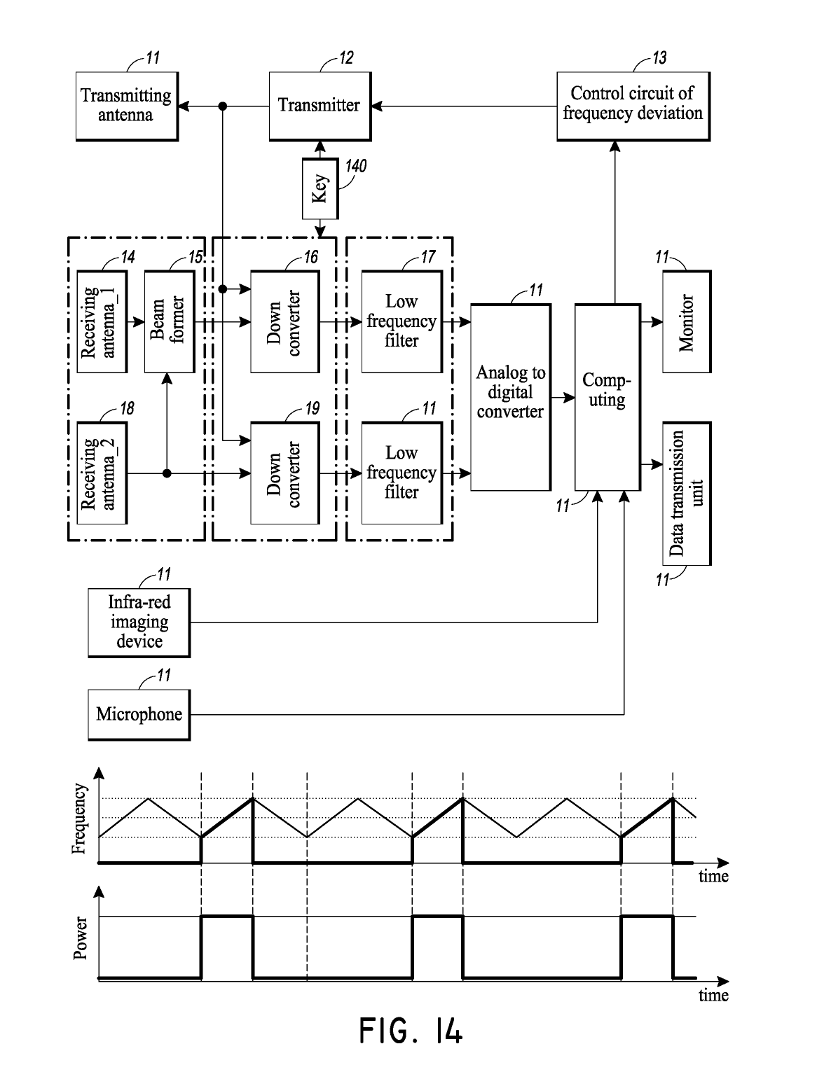

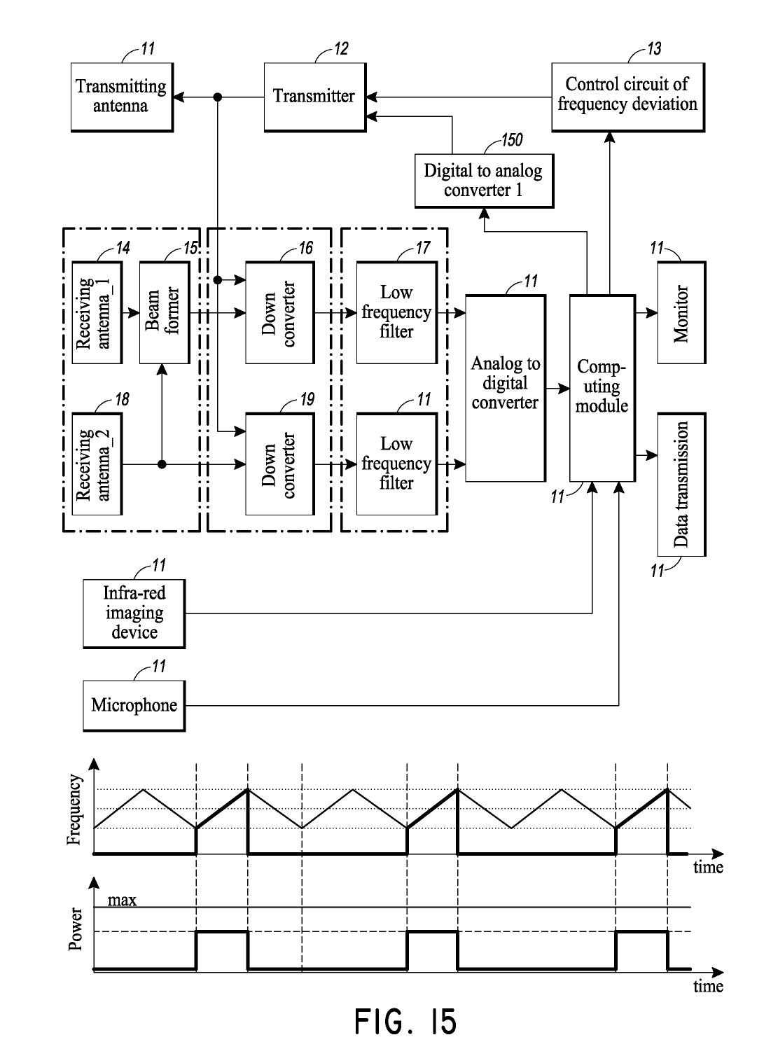

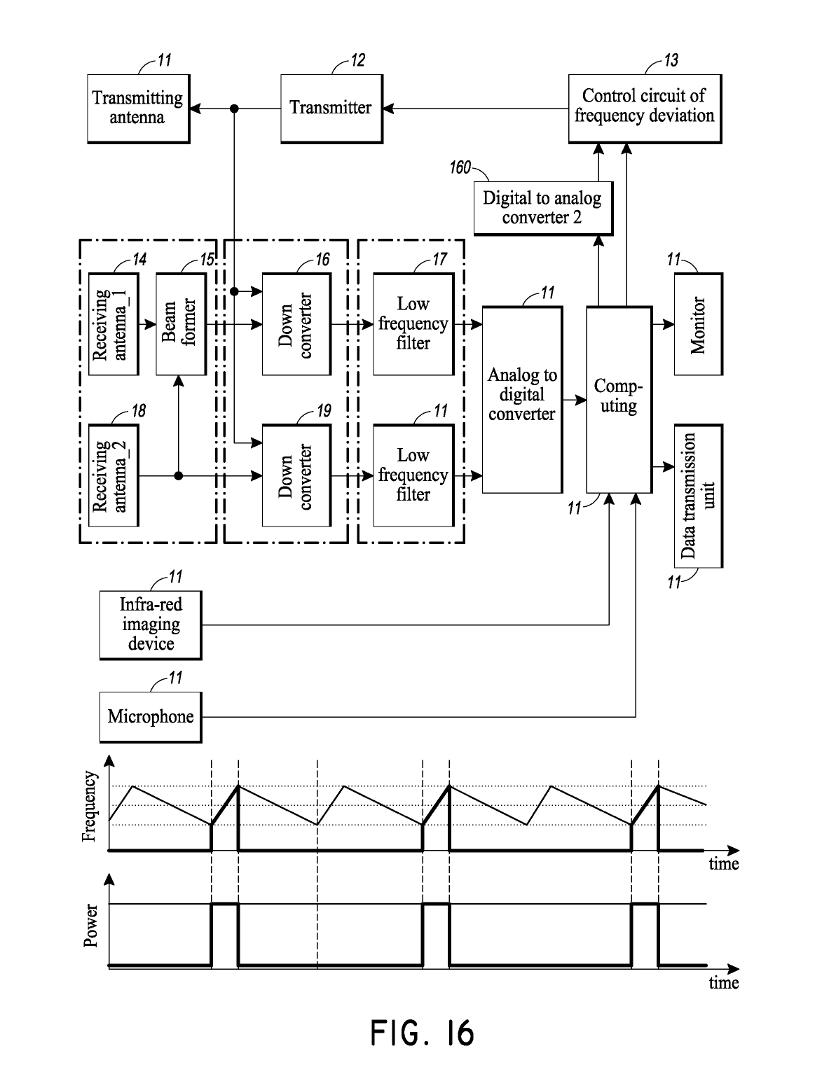

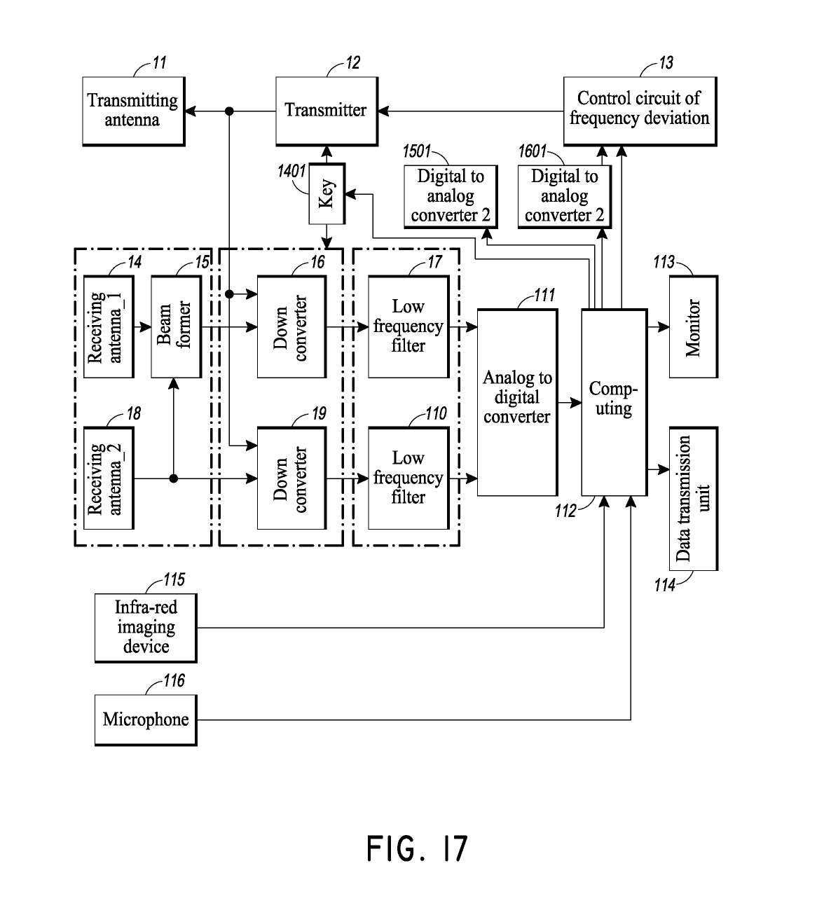

FIG. 12 illustrates typical signals that make it possible to identify limb movements. Limb movements are appearing as high frequency/high amplitude “noise” in the radar signal.

Optionally, the device can be equipped with an infrared imaging (IRI) device 115. IRI device can be used for monitoring body and limbs position of a subject during sleep. Moreover. IRI device can measure remotely the temperature variation of air flow in front of lips and nose, thus allowing to record respiratory phases in an additional channel. The information obtained with help of IRI device can be used in the analysis of readings of the claimed device.

Optionally, the device can be equipped with a microphone 116, which can be used to record snoring phases. Sleep apnea is often accompanied by snoring; therefore, the information acquired with help of the microphone can be used in the analysis of readings of the claimed device.

Diagnostics of sleep apnea condition is often aimed at providing a therapeutic effect on the patient. The claimed device and methods can be used in comprehensive sleep apnea therapy. In such case, a comprehensive therapy system can include a microwave radar-based device and a CPAP/BiPAP (Continuous Positive Airway Pressure/Bi-level Positive Airway Pressure) or similar device. If necessary, the system can include accelerometers and a pulse oximeter (FIG. 11).

The therapy system makes it possible to respond to changes in patient's condition in the real time mode and record data for subsequent post-processing.

The data about variations of breathing and respiration rate, amplitude of body fluctuations and plethysmogram parameters acquired using the claimed device can be used in the real time mode to control operating parameters of a CPAP/BiPAP or similar device. Additionally, blood saturation information from a pulse oximeter can be used.

Accelerometer data is used to get information about the position and motion of patient's body, which is important for post-processing of monitoring data.

General principles of remote contactless detection of sleep apnea based on subject's RCS data can be implemented not only in an FM radar-based device, but also in devices based on other radar types, such as the UWB radar and Doppler radar.

For a Doppler radar system, a known frequency signal is transmitted from an antenna, which is pointed at a reference object. A separate antenna is used to receive the signal that is reflected back from the reference to measure the Doppler shift of the signal.

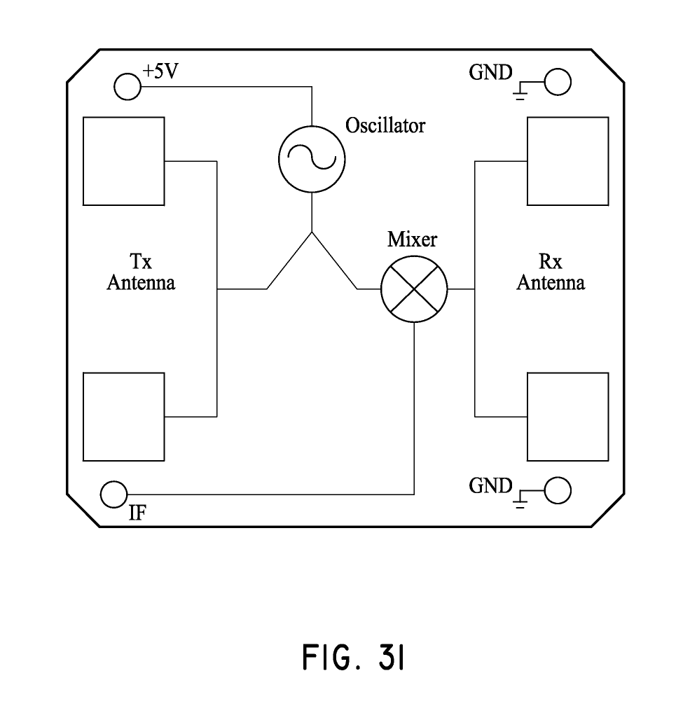

A simple Doppler module (see FIG. 31), also called a microwave motion sensor, can be easily integrated into the system of invention. Doppler modules have an internal oscillator used to produce the signal frequency transmitted as the source. The received signal is then mixed with this signal, which produces an output that is a sinusoid containing the frequency difference between the transmitted and received signals.

Ultra Wideband (UWB) radar systems transmit signals across a much wider frequency than conventional radar systems. The transmitted signal is significant for its very light power spectrum, which is lower than the allowed unintentional radiated emissions for electronics. The spectrum of a very narrow-width pulse has a very large frequency spectrum approaching that of white noise as the pulse becomes narrower and narrower. These very short pulses need a wider receiver bandwidth than conventional radar systems.

The amount of spectrum occupied by a signal transmitted by a UWB radar (i.e. the bandwidth of the UWB signal) is at least 25% of the center frequency. Thus, a UWB signal centered at 2 GHz would have a minimum bandwidth of 500 MHz and the minimum bandwidth of a UWB signal centered at 4 GHz would be 1 GHz. Often the absolute bandwidth is above 1 GHz.

In most systems, these values need to be recorded or read in a tangible way, and this is usually done with some sort of microcontroller. The easiest way for a microcontroller to read data from an analog device is if it outputs a DC level voltage. Some modules have this feature built into them. For those that do not, like the HB100 used in the ECE 480 Design Team 5 project, output just the AC signal. For these modules, a frequency-to-voltage circuit must be implemented. An IC, such as the LM2907N, can be used for this specific purpose or any other discrete component circuit. This circuit can be used to calibrate the output data for a specific set of expected frequencies coming from the module to contain it in reference voltage range.

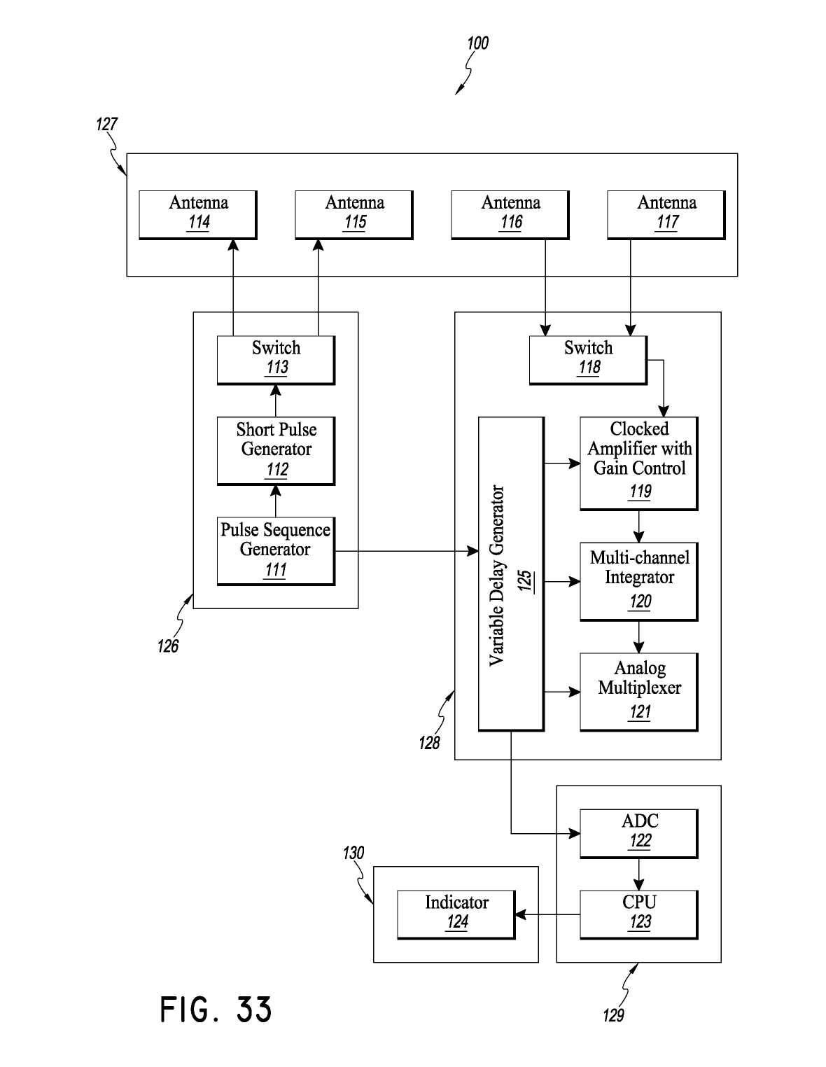

Further, an exemplary remote detection and monitoring apparatus (RDMA) is described, the apparatus being based on the UWB-radar technology. As shown in FIG. 33, RDMA 100 has the signal generation unit 126, the antenna unit 127, the signal processing unit 128, the device control unit 129, the display unit 130, and the power supply unit (not shown). The signal generation unit 126 includes the pulse sequence generator 111, the short pulse generator 112, and the switch 113. The antenna unit 127 includes a vertically polarized transmitting antenna 114, a horizontally polarized transmitting antenna 115, vertically polarized receiving antennae 116 and 117. The signal processing unit 128 includes the switch 118, the clocked amplifier with gain control 119, the multi-channel integrator 120, the analog multiplexer 121, and the variable delay generator 125. The device control unit 129 includes the analog-to-digital converter (ADC) 122 and the central processor (CPU) 123. The display unit 130 includes the display 124, or any other similar data display unit.

The pulse sequence generator 111 activates the short pulse generator 112 and sends the generated signal data to the variable delay generator 125 of the signal processing unit 128 for further signal processing. The switch 113 switches the transmission channels between transmitting antennae 114 and 115. In one embodiment, antennae 114 and 115 transmit a train of short pulses with a specific time delay generating an ultra wideband (UWB) signal. The transmitted signal is reflected by an object, and the return signal is received by receiving antennae 116 and 117. The return signal is fed to the clocked amplifier 119 having a gain control via the switch 118. The return signal is further processed by the multi-channel integrator 120 and the analog multiplexer 121.

The clocked amplifier 119 amplifies the received signal arrived from the switch 118. The amplifier input channel is activated only at a moment in time, allowing to receive return signals reflected from an object located at a certain distance range away from RDMA 100. The input channel opening signal is received from the variable delay generator 125. The multi-channel integrator 120 sums the signals received by each antenna over a specified time interval to achieve a better signal-to-noise ratio. The signals received by different receiving antennae are separated using the information from the variable delay generator 125. Analog multiplexer 121 provides the multichannel data to the ADC 122.

The clocked amplifier 119, the multi-channel integrator 120, and the analog multiplexer 121 receive the signal profile of the transmitted signal from the variable delay generator 125. Using the ADC 122, the return signal is converted to a digital signal, and the converted signal is delivered to the CPU 123 of the device control unit 129, where the return signal is processed. The processed data is delivered to the display 124 of display unit 130.

According to one embodiment, the antenna unit 127 may have only one antenna for both transmitting and receiving signals, or the antenna unit 127 may have one transmitting antenna 115 and one receiving antenna 116. With two receiving antennae 116 and 117, the azimuth of a target may be determined by a triangulation method. With the vertically polarized transmitting antenna 114, the horizontally polarized transmitting antenna 115, and two vertically polarized receiving antennae 116 and 117, richer information about the surface property and the condition of a target may be obtained to identify the target more accurately.

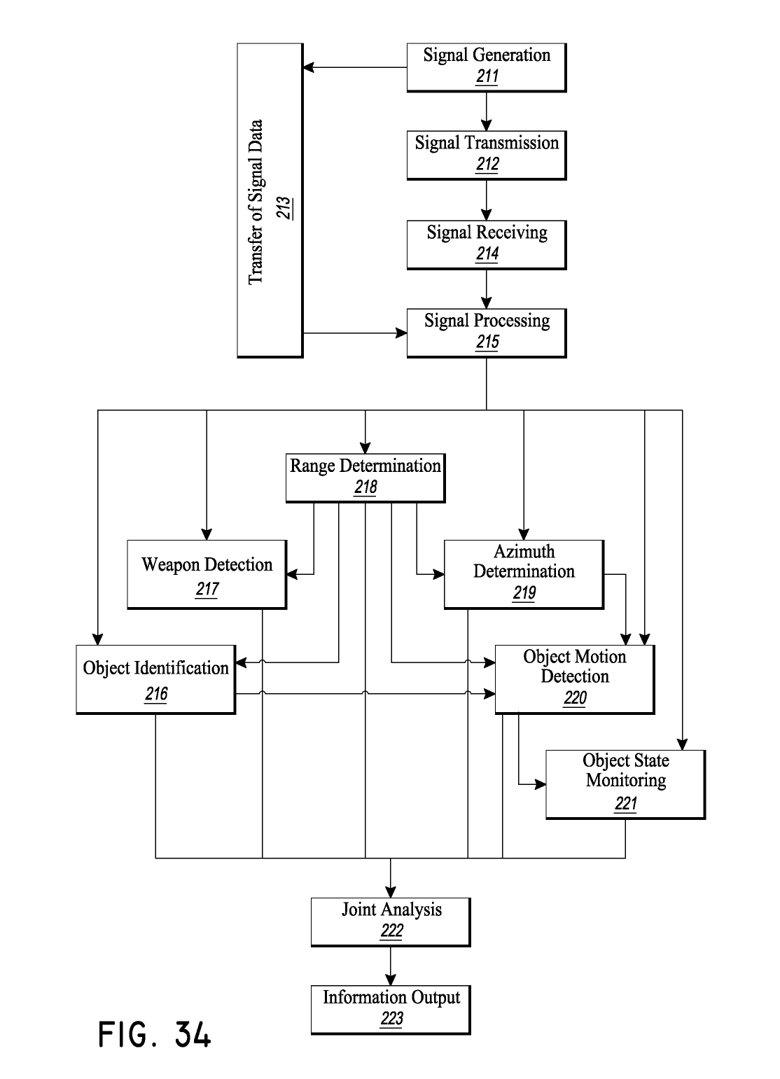

FIG. 34 illustrates an operational flow diagram of an exemplary RDMA, according to one embodiment. When the RDMA 100 is powered on, a probing signal is generated (211) and transmitted (212) using one or more transmitting antennae (e.g., 114 and 115). The return signal is received (214) by one or more receiving antennae (e.g., 116 and 117). The signal processing unit 128 receives and processes the transmitted signal data from signal generation unit 126 (213) as well as the return signal reflected from an object (or target). The signal processing unit 128 identifies the object (216) and detects whether the object is or contains a weapon and an explosive (217). Signal processing unit 128 also determines the distance to the object (218) and its azimuth 219. According to one embodiment, signal processing unit 128 monitors the return signal from an object over a period to determine whether the object is moving or motionless. According to another embodiment, the signal processing unit 128 determines the psycho-physiological attributes of a live object (221). Signal processing 215 may be performed by each individual process 216-221 or joint analysis may be performed (222) by congregating analysis results from each process 216-221. The processed data and information is displayed (223) using display 124 of the display unit 130.

Devices for medical use shall meet regulatory requirements as to subject exposure. An important goal for devices that require a subject to be exposed to energy radiated thereby is to decrease the radiated power to admissible levels.

To decrease the power radiated by the device, a controlled key 1401 is proposed to be included in the device's circuitry that will be switching power to the transmitter. The signal will then be radiated in pulses in the frequency rise stage with a periodicity every second cycle or more frequency modulation cycles.

Another modification of the device's circuitry aimed at decreasing its radiated power is to install a digital to analog converter 1501 between the computing module and the transmitter. This will enable programmable control of the radiated power in the range from minimum to maximum by issuing relevant commands to the digital to analog converter.

Yet another modification of the device's circuitry aimed at decreasing its radiated power is to install a digital to analog converter 1601 between the computing module and the control circuit of frequency deviations. This will enable programmable control of the radiated signal modulation frequency by issuing relevant commands to the digital to analog converter.

The proposed modifications of the device's circuitry can be used in aggregate, thus making it possible to significantly decrease the radiated power without affecting the device's performance. FIG. 17 illustrates the block diagram of the device, in which a controlled key 1401 for power switching to the transmitter, a digital to analog converter 1501 between the computing module and the transmitter, and a digital to analog converter 1601 between the computing module and the control circuit of frequency deviations are installed all together.

Further, an alternative embodiment of an apparatus for remote contactless detection of temporary cessation of breathing during sleep is described. The apparatus is generally referred here as SleepID device. It shall be clear that any known radar technologies, such as FM, Ultra Wide Band (UWB) or Doppler can be used for the purposes of the invention.

The SleepID device is designed for non-contact acquisition of patient data and for determination of apnea occurrences and associated snoring and desaturation occurrences in the patient's body.

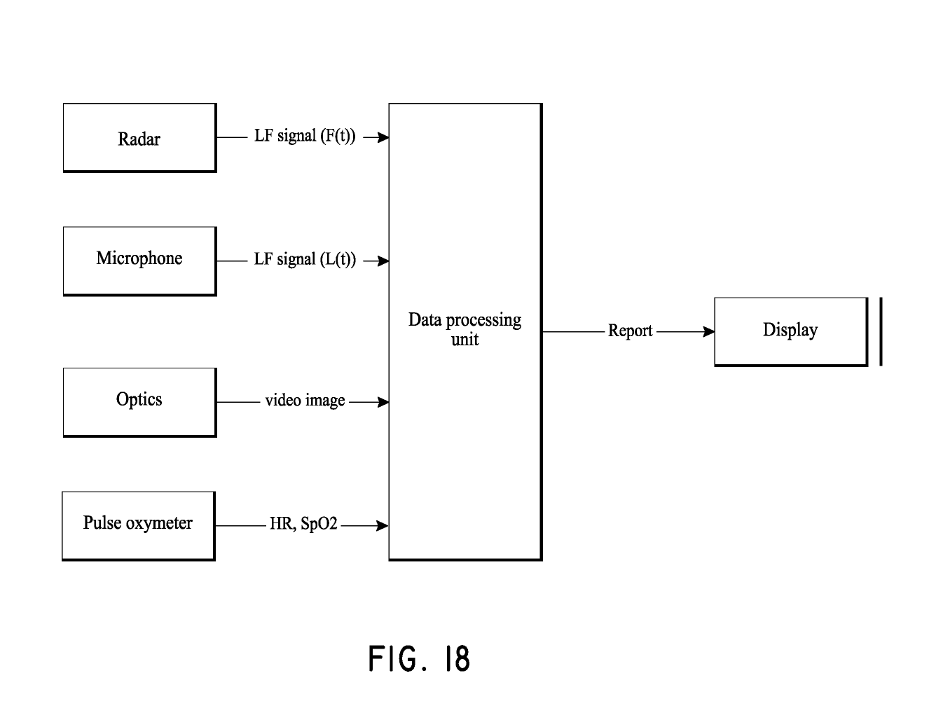

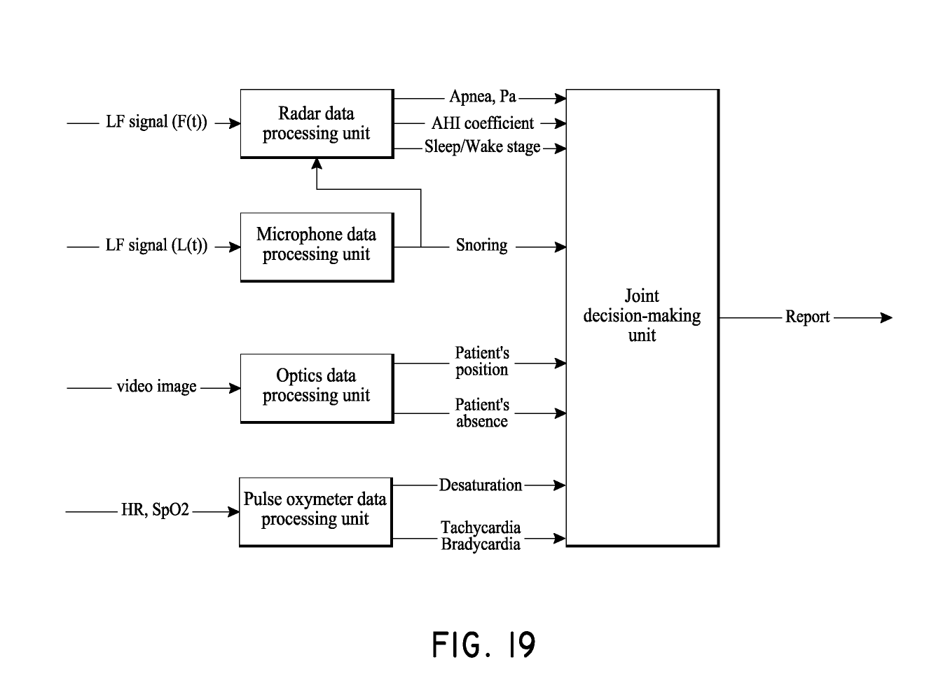

FIG. 18 shows the structure of SleepID. The device comprises the following main components: a radar, a microphone, an optic camera (optics), a pulse oximeter. The device is operated as follows. A patient with a wireless pulse oximeter sensor mounted on his finger lies on a bed at a distance of 2 to 3 meters from the device. The device radar emits the radio signal and receives a reflected signal containing patient's motion data, including motion of thorax, head, arms, legs and other parts of the body. The radar transmits a low-frequency signal (LF) (F(t)) to a data processing unit. Other sensors wirelessly transmit the following data to the processing unit:
low-frequency (LF) signal from the microphone (L(t));
video image feed from the optics;
heart rate (HR) and blood oxygen levels from the pulse oximeter.

Based on the received input data, the data processing unit determines apnea, snoring and desaturation occurrences, and registers mutual influence there between. A final report containing data regarding the extracted apnea, snoring and desaturation values, is delivered to the display of the device.

FIG. 19 shows the data processing unit structure. The operation of the data processing unit is further described.

The radar data processing unit receives the input LF-signal from the radar, extracts patient's thorax motion data therefrom, determines apnea occurrences (Apnea) based on said thorax motion data, evaluates apnea determination confidence (Pa) and determines average values for the determined apnea occurrences (AHI coefficient). Furthermore, based on the determined time intervals between the motions of the patient's body parts (arms, legs, head), the motion level and current state (Sleep/Wake) of the patient are determined. The Sleep/Wake state is corrected based on the snoring occurrences determined by the data processing unit based on the data received from the microphone. The obtained parameters are delivered to the joint decision-making unit.

The microphone data processing unit receives the LF-signal (L(t)) from the microphone, extracts snoring occurrences therefrom, and sends the corresponding data to the radar data processing unit and to the joint decision-making unit.

The optics data processing unit receives the video signal from the optical video camera and calculates the following two main factors based thereon:

a) the patient's presence in bed or his absence; for example, the patient could get up and temporarily leave the room;
b) the patient's sleeping position in bed.
The obtained factors are sent to the joint decision-making unit.

The pulse oximeter data processing unit receives heart rate (HR) and blood oxygen saturation (SpO2) values from the pulse oximeter. The unit provides calculation of desaturation level, tachycardia and bradycardia based on the input data. The obtained factors are sent to the joint decision-making unit. The joint decision-making unit registers mutual influence between the main parameters (apnea, snoring and desaturation) and delivers normalized values thereof to the display in the form of a final report.

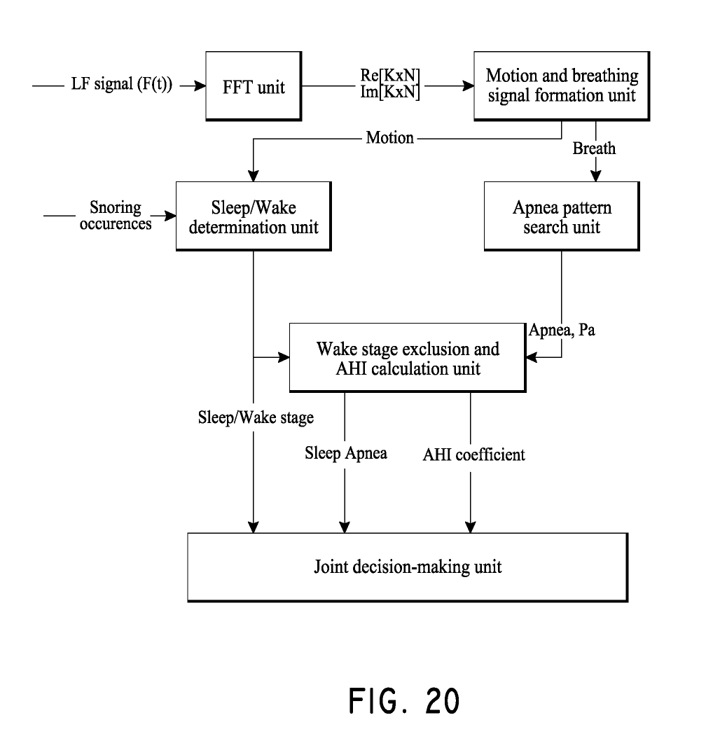

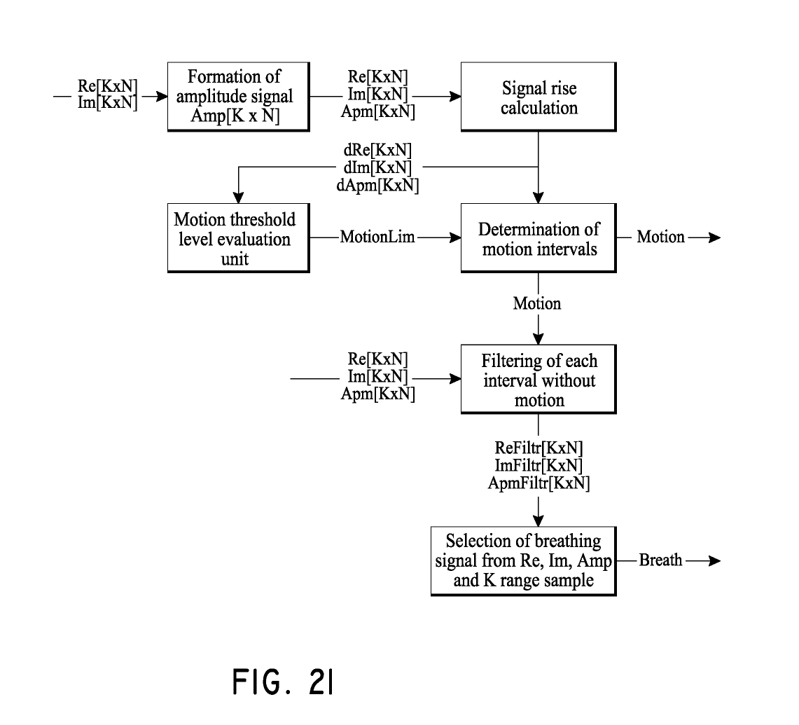

The structure of the radar data processing unit is shown in FIG. 20. The unit is operated as follows.

The digitized LF radar signal is fed to an FFT unit, in which the harmonic components Re, Im are calculated. The digitized signals whose samples correspond to a predetermined range rate are transmitted to a motion and breathing signal formation unit.

The motion and breathing signal formation unit determines time intervals, in which motion (Motion) related to limb motion, head motion or torso rotation is detected, and these intervals are transmitted to a Sleep/Wake determination unit. The signals corresponding to time intervals without motion are passed through a band-pass filter, and are used to form a Breath signal that most accurately corresponds to the motion of patient's thorax. The Breath signal is transmitted to an Apnea pattern search unit.

The Sleep/Wake determination unit calculates motional energy, which is used to determine the Sleep/Wake stages. To this effect, one or more threshold levels of motion are determined as averaged values over the complete monitoring period. From the relationship between the averaged motion value and the earlier determined threshold levels the status of the current interval: Sleep or Wake is determined.

The Apnea pattern search unit selects the breath amplitude signal and searches for patterns of apnea occurrences, most typical breathing disorders. The breath amplitude signal undergoes the following processing stages:
rough detection of apnea occurrences;
correction of the detected apnea occurrences by excluding occurrences, which go beyond the predetermined threshold levels by their duration;
removal of motion occurrences.

Data regarding the determined apnea occurrences (event start time, event duration) is transmitted to a Wake stage exclusion unit. The Wake stage exclusion and AHI calculation unit provides the following:
Exclusion of the Apnea occurrences during the Wake stage from the Apnea array;
Calculation of the AHI coefficient as Sleep apnea occurrences/sleep duration (hours)

The operational output of the radar data processing unit includes the Sleep/Wake stage, the Sleep Apnea array and the AHI coefficient. Said parameters are sent to the joint decision-making unit. FIG. 21 shows a schematic diagram of the motion and breathing signal formation unit.

Further, the unit operation process is described: Input data: Re[K×N]. Im[K×N], where N is the number of points, K is the number of samples. The amplitude signal formation unit calculates signal amplitude: Amp=\sqrt{Re²+Im²}.

The increment calculation unit determines the signal rise velocity modulus for each of the components:

$$d Re = \vert \frac{{Re}_{i + 1} - {Re}_{i}}{dt} \vert ,$$

etc. Said evaluation is performed for each signal (Re, Im, Amp) and for each sample according to range (N). (bogey value dt= 1/120).

Further, the motion threshold level evaluation unit is described. The motion threshold level (MotionLim) is a characteristic of separation of the breathing signal from the motion signal. Said level is calculated for the whole monitoring range as follows:

a) determination of mean rise values for each signal type (Re, Im, Amp) and for each range sample: mean(dRe), mean(dIm), mean(dAmp), where dRe, dIm, dAmp are K×N dimension arrays;

b) determination of the threshold level (MotionLim) as the maximum value of the mean rise values: MotionLim(N)=K*max (mean(dRe), mean(dIm), mean(dAmp), where K is the calibrating coefficient determined in a preliminary test of the method. K=1 by default.

Further, the motion interval extraction unit is described. The unit determines whether the extracted (current) monitoring interval belongs to Breath or Motion. The above is achieved by:

a) calculating the mean rise value for the current interval:

Motion_{—}int(n1)>=mean(dRe(n1−n2)),mean(dIm(n1−n2)),mean(dAmp(n1−n2));

b) comparing the obtained evaluations (for each signal and each sample) with the threshold value MotionLim(N):

if Motion_int(n1)>=MotionLim(N), the interval is marked as Motion, otherwise said interval is marked as Breath.

The subsequent processing (units 5 and 6, see below) is carried out for intervals marked as Breath (i.e. for intervals without motion): The filtering unit passes Re, Im, Amp signals of each range sample through a low pass filter, thins out the signals by lowering the sampling rate down to 10 Hz, and them applies a high pass filter.

The breath signal selection unit calculates the signal dispersion level for each filtered signal (Re, Im, Amp) according to each range sample. The signal with a maximum dispersion value is saved as the breathing signal (Breath) for the current time interval.

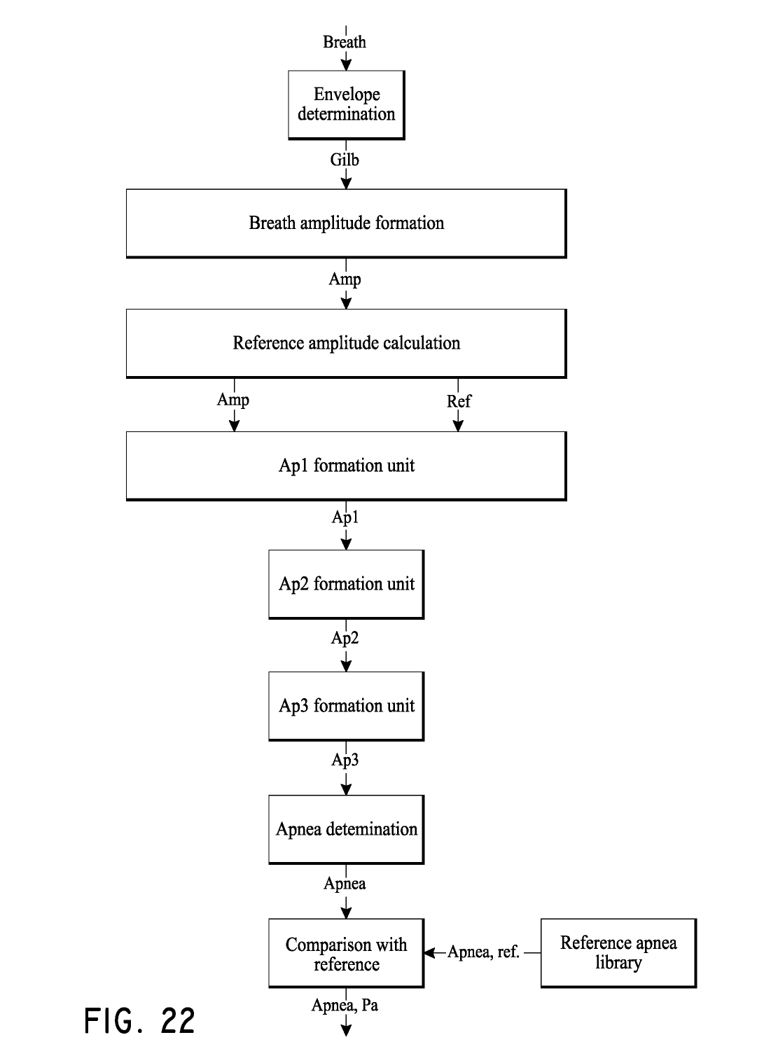

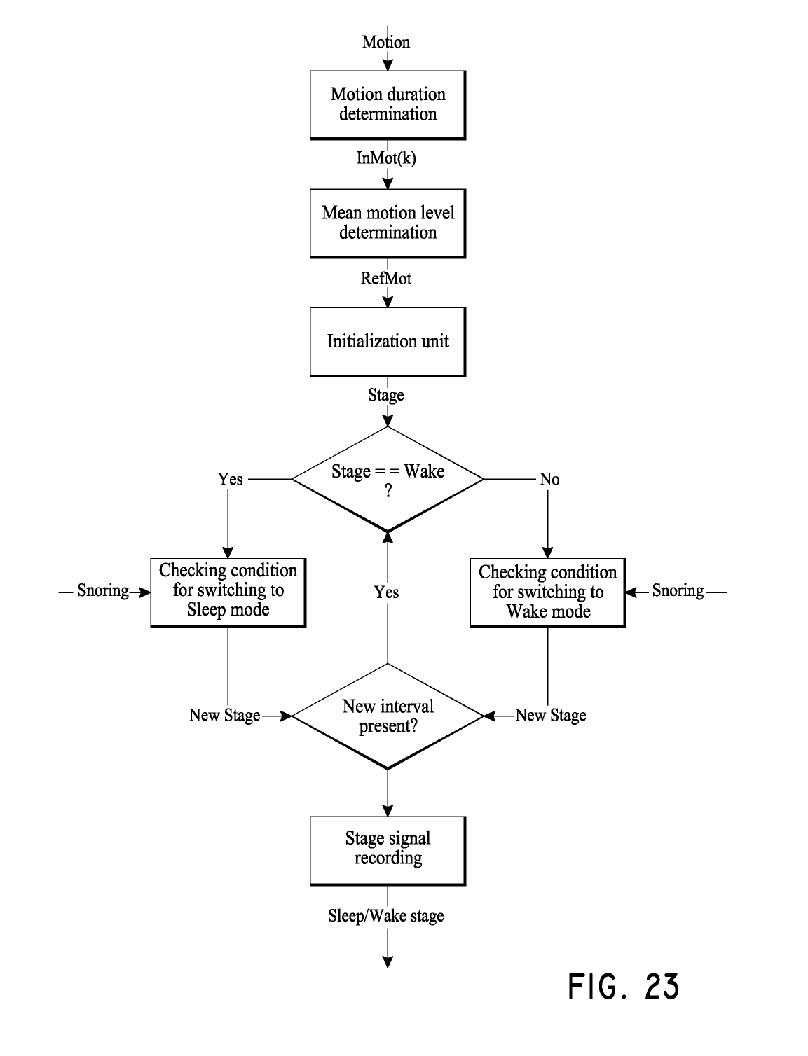

FIG. 22 shows a schematic diagram of the pattern search and apnea determination unit. Further the unit operation process is described:

The input signal envelope determination unit provides envelope determination based on the Hilbert transform:

$$H ( u ) ( t ) = - \frac{1}{π} {lim}_{ε -> 0} {\int }_{ε}^{∞} \frac{u ( t + τ ) - u ( t - τ )}{τ} d τ$$

The breath amplitude formation unit provides smoothing of the envelope values obtained in the previous step by interpolating envelope values between local maxima.

Further, the reference amplitude calculation unit is described. The unit provides calculation of the reference breath amplitude (Ref), and the apnea determination is later carried out with respect thereto. Ref value is calculated for the current interval between successive motions. In this case, Ref=K1*mean(Amp) or Ref=K2*max(Amp). K1 and K2 values in this case are chosen based on results of preliminary tests and the subsequent comparison of obtained results with reference device readings.

The Ap1 formation unit: determination of potential apnea occurrences. Ap1, Ap2, Ap3 are signals used for determining apnea patterns (intermediate potential apnea occurrences). Subsequent corrections are then made to determine whether said signals correspond to apnea occurrences. Ap1 is the result of Ref amplitude analysis. In this case, Ap1=1 if current breath amplitude exceeds a certain threshold: Amp>K3*Ref, Ap1=−1 if Amp<K4*Ref. In all other cases: Ap1=0. The operational output of the unit comprises presenting Ap1 in a square waveform (+1, 0, −1). In this case, <<−1>> denotes the apnea occurrence interval.

The Ap2 formation unit: Ap2=Ap1. The unit provides rejection of overly long potential apnea intervals with the value less than <<1>> (<<0>> or <<−1>>) and the duration of over 60 seconds. The operational output of the unit allows increasing apnea determination confidence.

The formation unit: Ap3=Ap2, with the exception of motion intervals. For motion intervals, Ap3=1. The unit provides removal of motion intervals.

The Apnea determination unit: Apnea=Ap3. with the exception of intervals, in which Ap3<1 and the duration of which is less than 10 seconds (the minimum apnea duration). For said intervals, Apnea=1. The formed Apnea signal indicates:

presence of clear patterns characteristic for breathing disorders: if the value is <<−1>>);

presence of less clear apnea patters (with lower confidence): if the value is 0.

The reference comparison unit compares the breathing signal (Breath) for a time interval corresponding to the determined Apnea occurrence with an array of reference apnea occurrences selected from a reference apnea library. The library is composed based on test measurements, in which the results of apnea determination based on the Breath signal are compared with readings from reference devices, which determine various apnea types. The so-called “golden standard of somnology” can be used as said reference devices. By comparing the current Breath signal with reference apnea occurrences, the match rate with each of the reference occurrences is determined. The possibility (Pa) that the determined event is an apnea occurrence is determined based on the maximum match rate value of all match rate values.

FIG. 23 shows the structure of the Sleep/Wake determination unit. The complete monitoring period is split into N-minute long intervals. Based on the analysis of the input Motion signal for each current interval k, the patient's motion time for the said interval is determined: InMot(k).

The mean motion level determination unit determines the mean motion time over the complete monitoring period: RefMot. Based on RefMot and the initial calibration, two parameters are determined: K1, K2 (K1<K2). In this case,

K1*RefMot is a threshold value for switching to a Sleep mode;

K2*RefMot is a threshold value for switching to a Wake mode.

The initialization unit. For the first several minutes of the recording, the Stage value is set as equal to the Wake value.

Condition for switching to Sleep mode. If InMot<K1*RefMot OR several snoring occurrences are present, then Stage=Sleep. Additionally, the video camera data regarding the patient's presence in bed is taken into account.

Condition for switching to Wake mode. If snoring occurrences are not present AND InMot>K2*RefMot, then Stage=Wake.

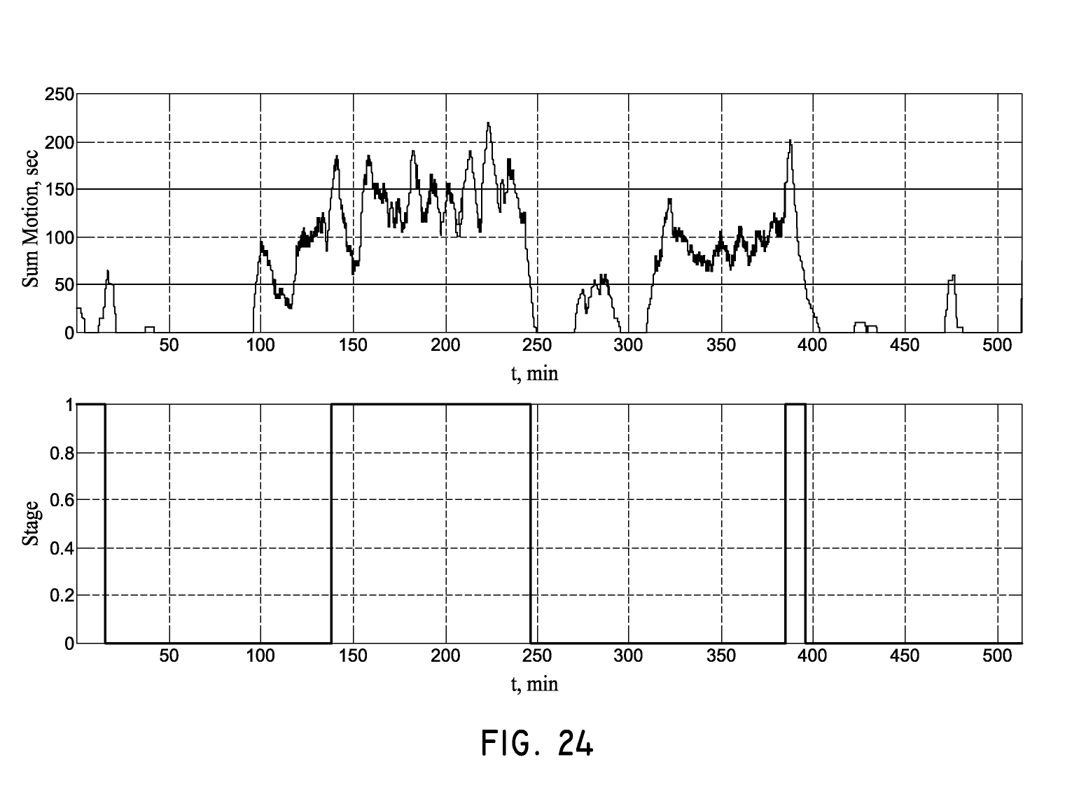

The operational results of units 4 and 5 are shown in FIG. 24 as determination of Sleep and Wake Modes. The upper graph represents the InMot(k) signal formed based on the unit 1 (see above FIG. 7). The lower graph represents Wake (=1) and Sleep (=0) signals obtained in accordance with processes described with the reference to units 4 and 5. The upper green line of the upper graph corresponds to K2*RefMot value, and the lower red line corresponds to K1*RefMot value. The condition check is performed until all N-minute intervals of the recording have been fully reviewed.

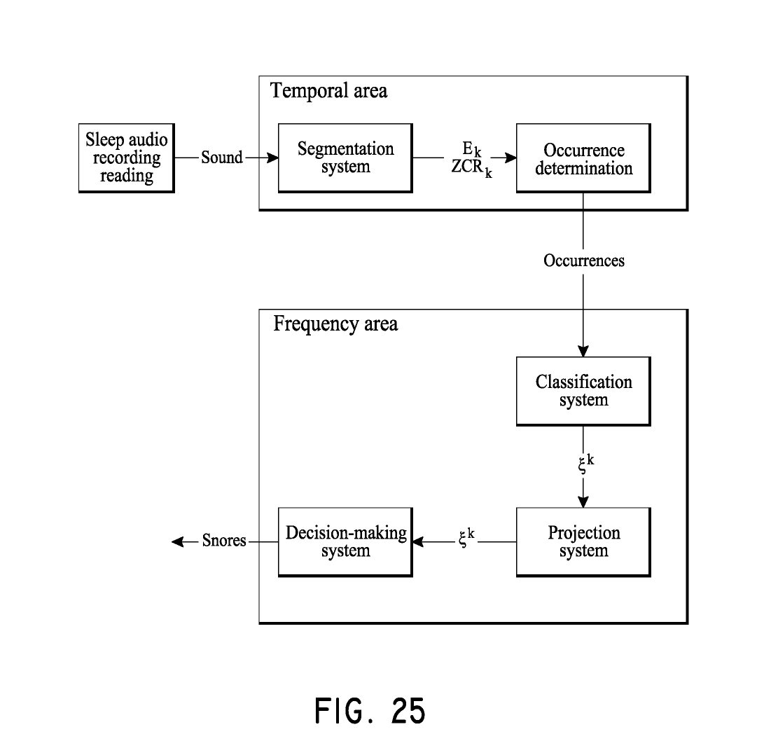

The structure of the microphone data processing unit is shown in FIG. 25. Following is the description of the flow chart functional units.

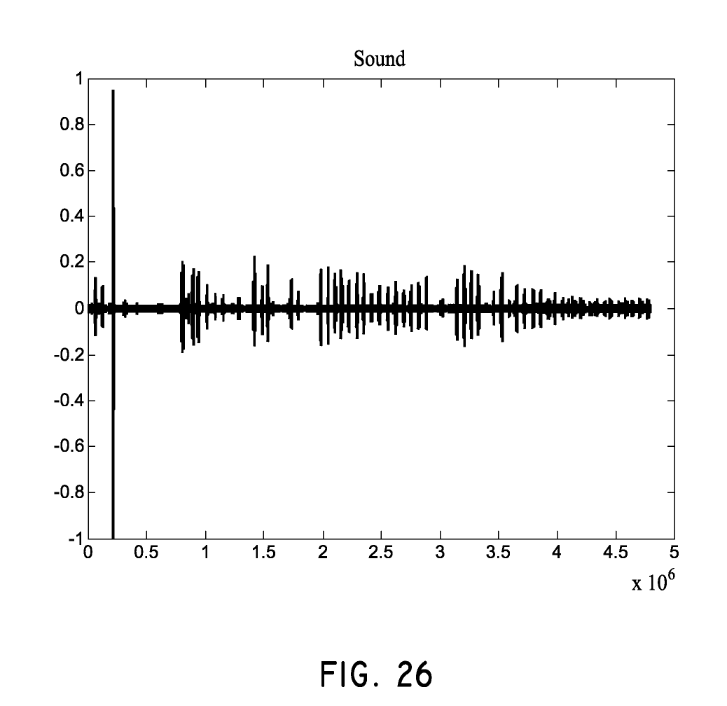

The sleep audio recording reading unit reads the input audio recording data; a typical audio recording is shown in FIG. 26.

The segmentation system unit. The input recording is segmented into frames (k) with intervals ΔT1 (with the typical value of 100 ms) and overlap ΔT2 (with the typical value of 50 ms)−(sk). For each segment, the following values are calculated:
signal energy (Ek): Eₖ=Σᵢ₌₀^{N-1}Sₖ²[i], where N is the number of segmented frames;
zero crossing number ZCRk): zCRₖ=Σᵢ₌₀^{N-2}I{Sₖ[i]·Sₖ[i+1]<0}, where |{Sₖ[i]·Sₖ[i+1]<0} is the zero crossing indicator.

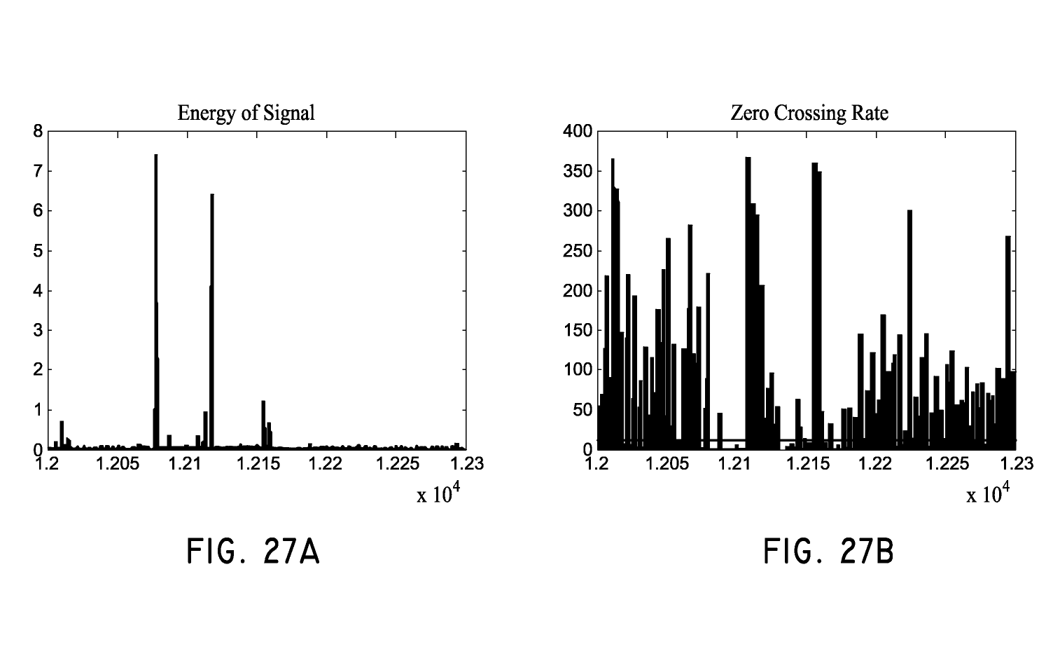

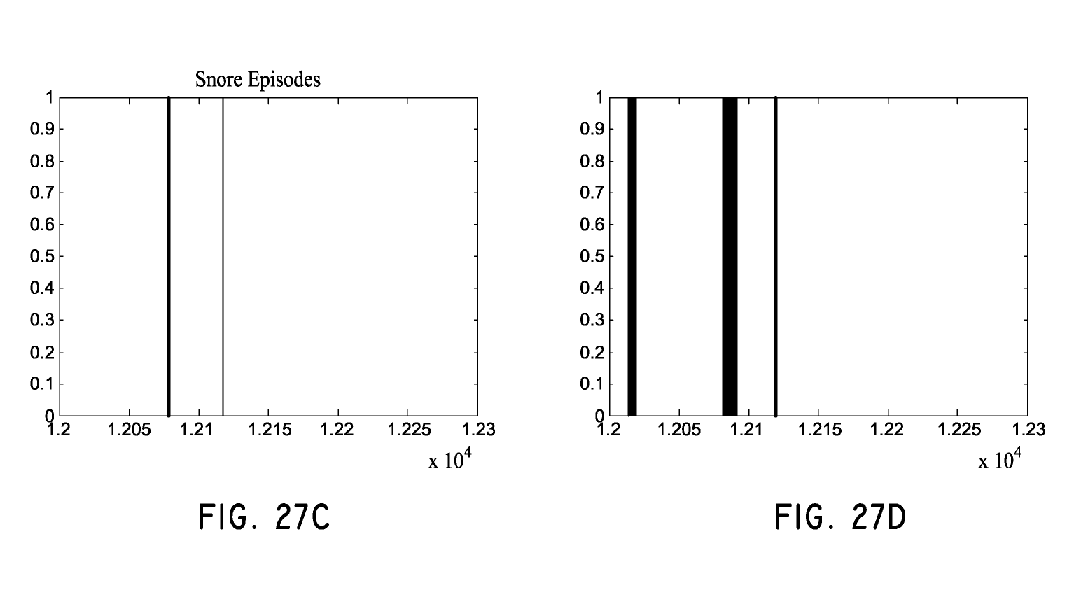

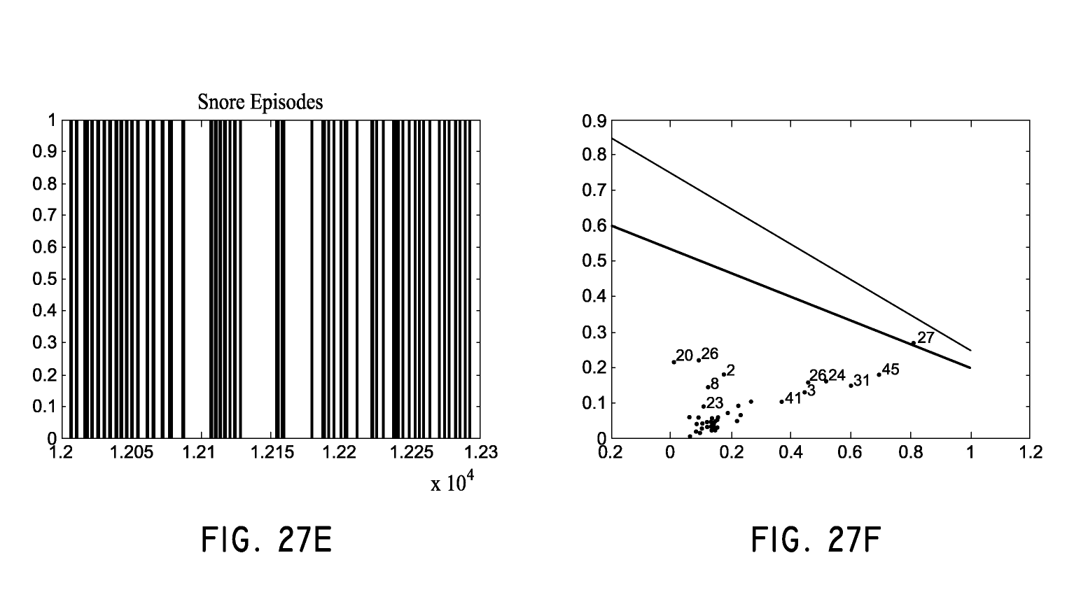

Examples of typical graphs are shown below in FIG. 27 (graphs 1-2).

The occurrence determination unit determines potential snoring occurrences. For this purpose, energy threshold (Te) values and zero crossing number (Tz) are determined: Tₑ=min(I₁,I₂), where

I₁=a·[max(Eₖ)−min(Eₖ)]+min(Eₖ),

I₂=b·min(Eₖ);

T_{Z}=mean(ZCRₖ)

Constant values a, b, c are determined experimentally using test data. Occurrence determination (for potential snoring occurrences) is performed using the following condition: if Eₖ>Tₑ AND ZCRₖ>T, then the frame represents a snoring occurrence.

A typical example of occurrence determination is shown below in FIG. 27 (graph 3).

The classification system unit: The present process comprises obtaining a vector of characteristic values determining weight (contribution) of the present spectral region in the entire occurrence spectrum. For this purpose, a spectrogram of each occurrence is calculated in the frequency range between 0 and 8000 Hz using a window of 256-point fast Fourier transform (FFT). The entire frequency range (0-8.000 Hz) is split into segments of 200 Hz each.

The vector of characteristic values (ξ) for a k-th occurrence is calculated as:

$${ξ}_{i}^{k} = \frac{{\sum }_{j = 1}^{{N}_{k}} {\sum }_{f = 200 ( i - 1 )}^{200 i} {\vert y ( j , i ) \vert}^{2}}{{\sum }_{j = 1}^{{N}_{k}} {\sum }_{f = 0}^{8000} {\vert y ( j , i ) \vert}^{2}} ,$$

where i=1, 2, . . . , 40 y(j, f) is signal power density for the j-th frame, and Nk is the number of frames in k-th occurrence.
The projection system unit multiplies the vector of characteristic values (ξ^{k}) by an optimized covariance matrix (Wₒₚₜ) obtained as follows:
Wₒₚₜ=arg_{w}maxdet(W^{T} CW), where C is the covariance matrix for the vector of characteristic values (ξk) of all snoring occurrences (K) in a test sample:

$$C = \frac{1}{K} {\sum }_{k} ( {ξ}^{k} - \overline{ξ} ) {( {ξ}^{k} - \overline{ξ} )}^{T} ,$$

where ξ̅ is the mean value of the vector of characteristic values in a test sample.
A two-dimensional coordinate vector of a projection of the vector of characteristic values onto the obtained subset of two highest characteristic values of the covariance matrix (C) is thus obtained:

ξ^{*}^{ₖ}=Wₒₚₜξ^{k}

The decision-making system unit determines whether the occurrence is a snoring occurrence (Sores) using the projection coordinate vector, see FIG. 27 (graph 6). The affiliation of vector coordinates situated between the lower and the upper threshold of the snoring area is thus determined.

FIG. 27 (graph 4) shows the process of determination of occurrences situated within the range of snoring subset.

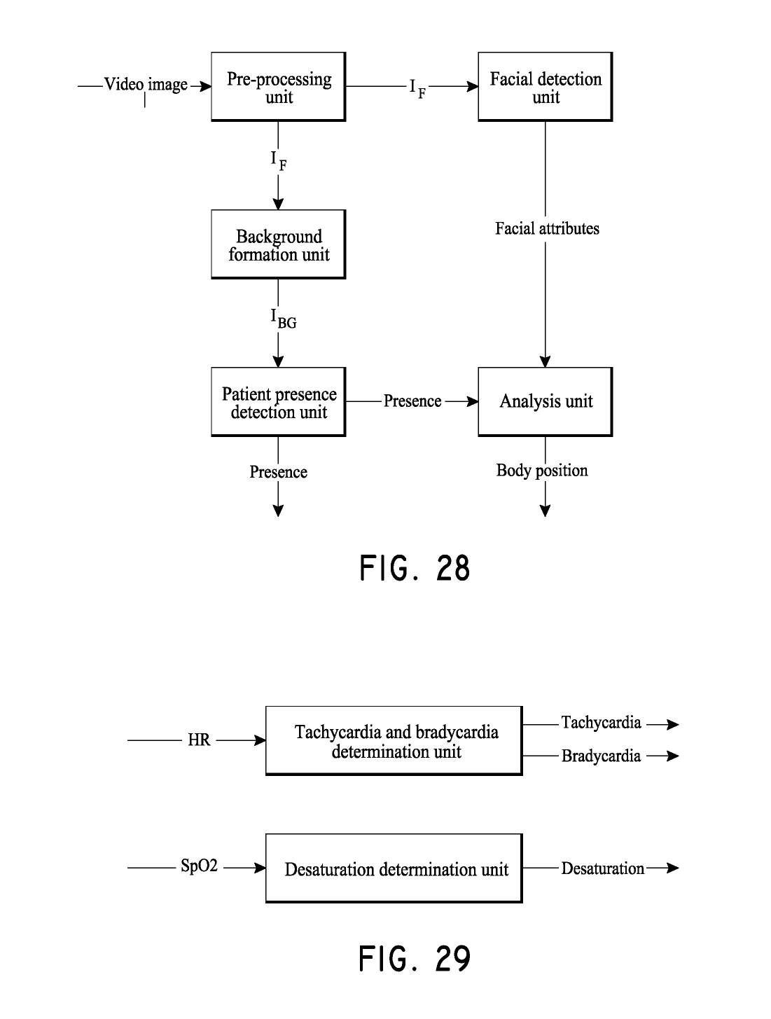

The structure of the video camera data processing unit is shown in FIG. 28. The unit is used to determine the following two main parameters based on the optics data:

a) patient's presence or absence i.e. the patient could leave for some time);

b) patient's body position (lying on his back, on one side, etc.).

Following is the functional description of units comprising the above structure. The purpose of the pre-processing unit is original image filtering, image contrast enhancement (to increase the possibility of image facial recognition).

Description: Median filtering is applied to the original image for antialiasing and pulse noise removal, thus obtaining image Im:

Iₘ(x,y)=median{I(i,j),(i,j)εw},

Median filter assigns a brightness value to each pixel (x,y), said brightness value being equal to a mean brightness value of a group of adjacent pixels, where w is the group of adjacent pixels surrounding (x,y).

The subsequent image editing procedure allows to uniformly enlarging the dynamic range of brightness levels, which leads to contrast enhancement of the output image. For this purpose, the density the of probability distribution of image Im is calculated for each intensity level k in the range [0,L−1], where L is the maximum intensity level: pₘ(k)=nₖ/n, where nk is the number of dots in the image with intensity k, and n is the total number of dots. The transformation function is calculated as:

$${s}_{k} = {\sum }_{j = 1}^{k} \frac{{n}_{j}}{n} , k = 0 , 1 , 2 , … , L - 1 , k = 0 , 1 , 2 , … , L - 1$$

The processed image (IF) is obtained by mapping each input pixel of the image with intensity k on an output image element with intensity sk:

I_{F}(x,y)=sₖ, if Iₘ(x,y)=k

The result is the filtered image IF.

The purpose of the background formation unit is building image background without the patient (IBG) necessary for operation of the patient presence detection unit. The unit stores video images (IF) of the background interior without the patient over a time interval t. Mean background value is then calculated:

mean

$$( {I}_{F} ) = \frac{1}{{N}_{t}} {\sum }_{i = 1}^{t} {I}_{F} ( i ) ,$$

where Nt is the number of received frames over the time interval t, and the mean-square deviation:

$$std ( {I}_{F} ) = \sqrt{\frac{1}{{N}_{t}} {\sum }_{i = 1}^{t} ( {I}_{F} ( i ) - {mean ( {I}_{F} )}^{2}}$$

A static background image (IBG) is formed using said parameters, with dynamically changeable dots excluded therefrom to increase the accuracy of subsequent patient detection: I_{BG}(x,y)=maxₜ{I_{F}^{t}(x,y)}, where |I_{F}^{t}(x,y)−mean(I_{F})|<2*std(I_{F}), I_{F}^{t}(x,y), is the intensity of dot (x,y) in the time interval t. The result is the static background image without the patient (I_{BG}).

The purpose of the patient presence detection unit is detecting the presence of the patient on the image using the static background image. The background image (IBG) is subtracted from the current frame (IF), and a binary mask (B) of foreground objects is calculated for the resulting image using threshold T: B(x,y)=1, if |I_{F}−I_{BG}|>T; 0 otherwise

The obtained binary mask is filtered by means of morphological operations using a structural element in the form of a disc having a diameter of d dots (D=DISK(d)).
The following morphological operations are subsequently applied:

<<erosion>> ((B⊖D={zεB|⊂B}) to remove static;

<<development>> (B⊕D=U_{zεD}B(z)) to eliminate outline tearing.

The area and the center-of-mass coordinate are calculated for the obtained outlines, and presence or absence of the patient on the image can be determined by comparing said parameters with threshold values. The result is the evidence of presence or absence of the patient.

The purpose of the facial detection unit is detection of faces on the image, as well as characteristic features thereof (position, tilting angle). The unit performs detection of faces on the image using the Viola-Jones algorithm. The algorithm detects faces on the image using classifiers obtained during a training step with reference data. In order to determine the facial shape and tilting angle, the classifier uses a cascade of reference rectangular attributes (primitive attributes) also determined during the classifier training step.

The search is performed in the active area of the image by primitive attributes allowing to describe the detected face and characteristic thereof: rectangle i={x,y,w,h,a}, where x, y are coordinates of i-th rectangle center, w is the width, h is the height, and a is the tilting angle of the rectangle with respect to vertical axis of the image. All obtained attributes are sent to the classifier that detects whether the image area depicts a face. The result is detected facial image and characteristic attributes (position, tilting angle).

The purpose of the analysis unit is to determine the patient's position in bed using data regarding patient's presence and facial position on the image. The analysis of current position of the patient is performed using facial data previously obtained by the facial detection unit according to the table below. Values in the first column: 0—the patient is absent from the image, 1—the patient is present in the image. Values in the second column: 0—face not found, 1—face found, tilting angle <30°, 2—face found, tilting angle >30°. Values in the third column: 0—the patient is absent, 1—the patient is lying on his back, 2—the patient is lying on his side.

TABLE 1
Determination of body position

| Presence/Absence | Face position | Body position |
|---|---|---|
| 1 | 0 | 2 |
| 1 | 1 | 2 |
| 1 | 2 | 1 |
| 0 | 0 | 0 |
| 0 | 1 | 0 |
| 0 | 2 | 0 |

The result is the position of patient in bed is determined.

FIG. 29 shows the structure of the pulse oximeter data processing unit. The unit performs the following functions: The tachycardia and bradycardia determination unit calculates tachycardia and bradycardia levels.

The desaturation determination unit calculates desaturation based on the blood oxygen saturation (SpO2) parameter received from the pulse oximeter. If the SpO2 level fulfills the condition: SpO2<P1 AND tn>T1, then the current time

interval (tn) is marked as desaturation. P1 and T1 threshold values are determined based on preliminary testing using reference devices.

The joint decision-making unit performs the following functions:

a) increasing apnea determination confidence due to joint tracking of apnea occurrences with snoring and desaturation occurrences;

b) correcting the AHI coefficient by taking into account the normalized apnea readings;

c) registering the correlation between apnea reading and the patient's position determined by processing data obtained by the video camera;

d) determining snoring attributes by taking into account the correlation between snoring and position of the patient's body;

e) Wake status correction by taking into account time intervals during which the patient is absent, said intervals determined by processing data obtained by the video camera;

f) correcting the desaturation status by taking into account Wake intervals.

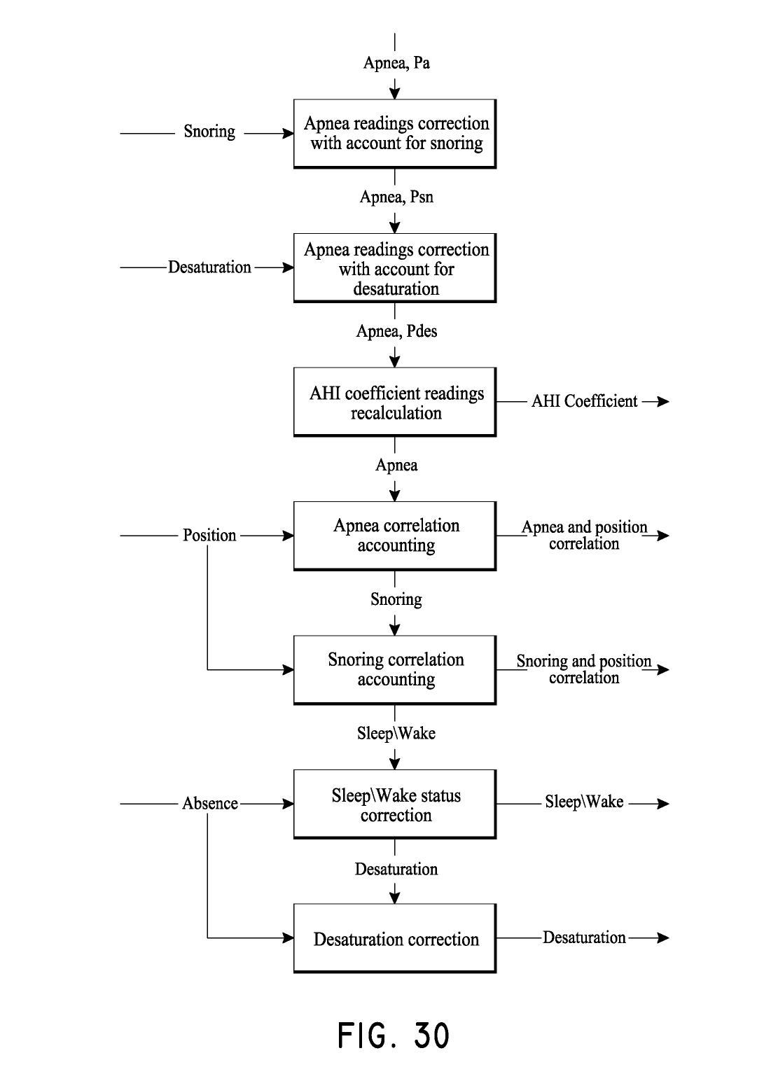

The structure of the joint decision-making unit is shown in FIG. 30. Description of unit operation process follows:

a. The unit of apnea readings correction with account for snoring corrects (increases confidence) apnea occurrences by taking into account the correlation between an apnea occurrence and a subsequent (delayed) snoring occurrence. The following process is thus performed for each previously detected apnea occurrence: confidence level for the determined apnea occurrence (Pa) is corrected (increased) based on the subsequent snoring occurrence taking into account the delay time interval between the onset of the snoring occurrence and the onset of the apnea occurrence. The final confidence value (Psn) is determined using a correlation matrix (between snoring and apnea occurrences). Coefficients of said correlation matrix are obtained by processing test measurements obtained using reference “gold standard” devices.

b. The unit of apnea readings correction with account for desaturation corrects (increases confidence) apnea occurrences (based on corrections obtained in the previous step (Psn)) by taking into account the correlation between an apnea occurrence and subsequent (delayed) desaturation occurrence. The following process is thus performed for each previously detected apnea occurrence: confidence level for the determined apnea occurrence (Psn) is corrected (increased) based on the subsequent snoring occurrence taking into account the delay time interval between the onset of the desaturation occurrence and the onset of the apnea occurrence. The final confidence value (Pdes is determined using a correlation matrix (between desaturation and apnea occurrences). Coefficients of said correlation matrix are obtained by processing test measurements obtained using reference “gold standard” devices.

c. The AHI coefficient readings recalculation unit corrects said coefficient by taking into account the corrections of Pdes values obtained for each determined apnea occurrence.

d. The apnea correlation accounting unit accounts for correlation of apnea with patient's position readings. A degree of correlation with determined apnea occurrences is determined for each position (lying on the back, on the side, etc.) of the patient's body.

e. The snoring correlation accounting unit accounts for correlation of snoring occurrences with patient's position readings. A degree of correlation with determined snoring occurrences is determined for each position (lying on the back, on the side, etc.) of the patient's body.

f. The Sleep/Wake status correction unit accounts for the occurrences of patient's absence: all occurrences of patient's absence are given Wake status.

g. The desaturation correction unit accounts for the occurrences of patient's absence: all occurrences of patient's absence are excluded from desaturation status.

1. An apparatus for remote monitoring of sleep apnea, comprising a radar transmitter having a transmitting antenna for radiation of radio frequency signal towards at least one human body, and at least one radar receiver for receiving a signal reflected from the at least one human body, the radar receiver comprising a receiving antenna positioned at a predefined distance from the transmitting antenna, the apparatus further comprising at least one accelerometer adapted to be placed on a human body, at least one microphone, at least one infrared imaging device, and a signal processor, wherein respective outputs of the radar receiver, accelerometer, microphone, and infrared imaging device are connected to the input of the signal processor which is configured for extracting and processing apnea-specific physiological parameters of the at least one human body from the inputted signals of the receiver, accelerometer, microphone, and infrared imaging device.

2. The apparatus of claim 1, wherein the accelerometer comprises a wireless transmitter, the signal processor comprises a wireless receiver, and the output of the accelerometer is connected to the input of the said signal processor through a wireless communication channel.

3. The apparatus of claim 1, comprising at least one optical video camera and at least one pulse oximeter, wherein the optical input of the video camera is directed to the at least one human body, the pulse oximeter is adapted to be positioned on the at least one human body, and the outputs of the video camera and pulse oximeter are connected to respective inputs of the signal processor.

4. The apparatus of claim 3, wherein the pulse oximeter comprises a wireless transmitter, and the output of the pulse oximeter is connected to the input of the said signal processor through a wireless channel.

5. The apparatus of claim 3, wherein the pulse oximeter is adapted to be positioned on a human finger.

6. The apparatus of claim 1, further comprising at least two radar receivers, wherein their respective receiving antennae are positioned at a predetermined distance from one another and from the transmitting antenna.

7. The apparatus of claim 1, wherein the radar receiver comprises a clocked amplifier having its input connected to the receiving antenna.

8. The apparatus of claim 1, wherein the transmitter is adapted to produce frequency modulated signals in the form of a train of pulses with a predefined delay between the pulses.
